SEQUENCE LISTING (27 sequences)

SEQ ID NO: 1 - protein, 1049 aa, human
MDWSFFRVVAVLFIFLVVVEVNSEFRIQVRDYNTKNGTIKWHSIRRQKREWIKFAAACREGEDNSKRNPIAKIHSDCAANQQVTYRISGVGIDQPPYGIFVINQKTGEINITSIVDREVTPFFIIYCRALNSMGQDLERPLELRVRVLDINDNPPVFSMATFAGQIEENSNANTLVMILNATDADEPNNLNSKIAFKIIRQEPSDSPMFIINRNTGEIRTMNNFLDREQYGQYALAVRGSDRDGGADGMSAECECNIKILDVNDNIPYMEQSSYTIEIQENTLNSNLLEIRVIDLDEEFSANWMAVIFFISGNEGNWFEIEMNERTNVGILKVVKPLDYEAMQSLQLSIGVRNKAEFHHSIMSQYKLKASAISVTVLNVIEGPVFRPGSKTYVVTGNMGSNDKVGDFVATDLDTGRPSTTVRYVMGNNPADLLAVDSRTGKLTLKNKVTKEQYNMLGGKYQGTILSIDDNLQRTCTGTININIQSFGNDDRTNTEPNTKITTNTGRQESTSSTNYDTSTTSTDSSQVYSSEPGNGAKDLLSDNVHFGPAGIGLLIMGFLVLGLVPFLMICCDCGGAPRSAAGFEPVPECSDGAIHSWAVEGPQPEPRDITTVIPQIPPDNANIIECIDNSGVYTNEYGGREMQDLGGGERMTGFELTEGVKTSGMPEICQEYSGTLRRNSMRECREGGLNMNFMESYFCQKAYAYADEDEGRPSNDCLLIYDIEGVGSPAGSVGCCSFIGEDLDDSFLDTLGPKFKKLADISLGKESYPDLDPSWPPQSTEPVCLPQETEPVVSGHPPISPHFGTTTVISESTYPSGPGVLHPKPILDPLGYGNVTVTESYTTSDTLKPSVHVHDNRPASNVVVTERVVGPISGADLHGMLEMPDLRDGSNVIVTERVIAPSSSLPTSLTIHHPRESSNVVVTERVIQPTSGMIGSLSMHPELANAHNVIVTERVVSGAGVTGISGTTGISGGIGSSGLVGTSMGAGSGALSGAGISGGGIGLSSLGGTASIGHMRSSSDHHFNQTIGSASPSTARSRITKYSTVQYSK

SEQ ID NO: 2 - protein, 2871 aa, human
MSCNGGSHPRINTLGRMIRAESGPDLRYEVTSGGGGTSRMYYSRRGVITDQNSDGYCQTGTMSRHQNQNTIQELLQNCSDCLMRAELIVQPELKYGDGIQLTRSRELDECFAQANDQMEILDSLIREMRQMGQPCDAYQKRLLQLQEQMRALYKAISVPRVRRASSKGGGGYTCQSGSGWDEFTKHVTSECLGWMRQQRAEMDMVAWGVDLASVEQHINSHRGIHNSIGDYRWQLDKIKADLREKSAIYQLEEEYENLLKASFERMDHLRQLQNIIQATSREIMWINDCEEEELLYDWSDKNTNIAQKQEAFSIRMSQLEVKEKELNKLKQESDQLVLNQHPASDKIEAYMDTLQTQWSWILQITKCIDVHLKENAAYFQFFEEAQSTEAYLKGLQDSIRKKYPCDKNMPLQHLLEQIKELEKEREKILEYKRQVQNLVNKSKKIVQLKPRNPDYRSNKPIILRALCDYKQDQKIVHKGDECILKDNNERSKWYVTGPGGVDMLVPSVGLIIPPPNPLAVDLSCKIEQYYEAILALWNQLYINMKSLVSWHYCMIDIEKIRAMTIAKLKTMRQEDYMKTIADLELHYQEFIRNSQGSEMFGDDDKRKIQSQFTDAQKHYQTLVIQLPGYPQHQTVTTTEITHHGTCQDVNHNKVIETNRENDKQETWMLMELQKIRRQIEHCEGRMTLKNLPLADQGSSHHITVKINELKSVQNDSQAIAEVLNQLKDMLANFRGSEKYCYLQNEVFGLFQKLENINGVTDGYLNSLCTVRALLQAILQTEDMLKVYEARLTEEETVCLDLDKVEAYRCGLKKIKNDLNLKKSLLATMKTELQKAQQIHSQTSQQYPLYDLDLGKFGEKVTQLTDRWQRIDKQIDFRLWDLEKQIKQLRNYRDNYQAFCKWLYDAKRRQDSLESMKFGDSNTVMRFLNEQKNLHSEISGKRDKSEEVQKIAELCANSIKDYELQLASYTSGLETLLNIPIKRTMIQSPSGVILQEAADVHARYIELLTRSGDYYRFLSEMLKSLEDLKLKNTKIEVLEEELRLARDANSENCNKNKFLDQNLQKYQAECSQFKAKLASLEELKRQAELDGKSAKQNLDKCYGQIKELNEKITRLTYEIEDEKRRRKSVEDRFDQQKNDYDQLQKARQCEKENLGWQKLESEKAIKEKEYEIERLRVLLQEEGTRKREYENELAKVRNHYNEEMSNLRNKYETEINITKTTIKEISMQKEDDSKNLRNQLDRLSRENRDLKDEIVRLNDSILQATEQRRRAEENALQQKACGSEIMQKKQHLEIELKQVMQQRSEDNARHKQSLEEAAKTIQDKNKEIERLKAEFQEEAKRRWEYENELSKVRNNYDEEIISLKNQFETEINITKTTIHQLTMQKEEDTSGYRAQIDNLTRENRSLSEEIKRLKNTLTQTTENLRRVEEDIQQQKATGSEVSQRKQQLEVELRQVTQMRTEESVRYKQSLDDAAKTIQDKNKEIERLKQLIDKETNDRKCLEDENARLQRVQYDLQKANSSATETINKLKVQEQELTRLRIDYERVSQERTVKDQDITRFQNSLKELQLQKQKVEEELNRLKRTASEDSCKRKKLEEELEGMRRSLKEQAIKITNLTQQLEQASIVKKRSEDDLRQQRDVLDGHLREKQRTQEELRRLSSEVEALRRQLLQEQESVKQAHLRNEHFQKAIEDKSRSLNESKIEIERLQSLTENLTKEHLMLEEELRNLRLEYDDLRRGRSEADSDKNATILELRSQLQISNNRTLELQGLINDLQRERENLRQEIEKFQKQALEASNRIQESKNQCTQVVQERESLLVKIKVLEQDKARLQRLEDELNRAKSTLEAETRVKQRLECEKQQIQNDLNQWKTQYSRKEEAIRKIESEREKSEREKNSLRSEIERLQAEIKRIEERCRRKLEDSTRETQSQLETERSRYQREIDKLRQRPYGSHRETQTECEWTVDTSKLVFDGLRKKVTAMQLYECQLIDKTTLDKLLKGKKSVEEVASEIQPFLRGAGSIAGASASPKEKYSLVEAKRKKLISPESTVMLLEAQAATGGIIDPHRNEKLTVDSAIARDLIDFDDRQQIYAAEKAITGFDDPFSGKTVSVSEAIKKNLIDRETGMRLLEAQIASGGVVDPVNSVFLPKDVALARGLIDRDLYRSLNDPRDSQKNFVDPVTKKKVSYVQLKERCRIEPHTGLLLLSVQKRSMSFQGIRQPVTVTELVDSGILRPSTVNELESGQISYDEVGERIKDFLQGSSCIAGIYNETTKQKLGIYEAMKIGLVRPGTALELLEAQAATGFIVDPVSNLRLPVEEAYKRGLVGIEFKEKLLSAERAVTGYNDPETGNIISLFQAMNKELIEKGHGIRLLEAQIATGGIIDPKESHRLPVDIAYKRGYFNEELSEILSDPSDDTKGFFDPNTEENLTYLQLKERCIKDEETGLCLLPLKEKKKQVQTSQKNTLRKRRVVIVDPETNKEMSVQEAYKKGLIDYETFKELCEQECEWEEITITGSDGSTRVVLVDRKTGSQYDIQDAIDKGLVDRKFFDQYRSGSLSLTQFADMISLKNGVGTSSSMGSGVSDDVFSSSRHESVSKISTISSVRNLTIRSSSFSDTLEESSPIAAIFDTENLEKISITEGIERGIVDSITGQRLLEAQACTGGIIHPTTGQKLSLQDAVSQGVIDQDMATRLKPAQKAFIGFEGVKGKKKMSAAEAVKEKWLPYEAGQRFLEFQYLTGGLVDPEVHGRISTEEAIRKGFIDGRAAQRLQDTSSYAKILTCPKTKLKISYKDAINRSMVEDITGLRLLEAASVSSKGLPSPYNMSSAPGSRSGSRSGSRSGSRSGSRSGSRRGSFDATGNSSYSYSYSFSSSSIGH

SEQ ID NO: 3 - protein, 211 aa, human
MISRMEKMTMMMKILIMFALGMNYWSCSGFPVYDYDPSSLRDALSASVVKVNSQSLSPYLFRAFRSSLKRVEVLDENNLVMNLEFSIRETTCRKDSGEDPATCAFQRDYYVSTAVCRSTVKVSAQQVQGVHARCSWSSSTSESYSSEEMIFGDMLGSHKWRNNYLFGLISDESISEQFYDRSLGIMRRVLPPGNRRYPNHRHRARINTDFE

SEQ ID NO: 4 - protein, 355 aa, human
MAKLIALTLLGMGLALFRNHQSSYQTRLNALREVQPVELPNCNLVKGIETGSEDMEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLQSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 5 - protein, 375 aa, human
MERASCLLLLLLPLVHVSATTPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPGTALPHEGSMNSGVVPACARSTLSVGVSGTLVLLQGARGFA

SEQ ID NO: 6 - protein, 1128 aa, human
MSRSVLQPSQQKLAEKLTILNDRGVGMLTRLYNIKKACGDPKAKPSYLIDKNLESAVKFIVRKFPAVETRNNNQQLAQLQKEKSEILKNLALYYFTFVDVMEFKDHVCELLNTIDVCQVFFDITVNFDLTKNYLDLIITYTTLMILLSRIEERKAIIGLYNYAHEMTHGASDREYPRLGQMIVDYENPLKKMMEEFVPHSKSLSDALISLQMVYPRRNLSADQWRNAQLLSLISAPSTMLNPAQSDTMPCEYLSLDAMEKWIIFGFILCHGILNTDATALNLWKLALQSSSCLSLFRDEVFHIHKAAEDLFVNIRGYNKRINDIRECKEAAVSHAGSMHRERRKFLRSALKELATVLSDQPGLLGPKALFVFMALSFARDEIIWLLRHADNMPKKSADDFIDKHIAELIFYMEELRAHVRKYGPVMQRYYVQYLSGFDAVVLNELVQNLSVCPEDESIIMSSFVNTMTSLSVKQVEDGEVFDFRGMRLDWFRLQAYTSVSKASLGLADHRELGKMMNTIIFHTKMVDSLVEMLVETSDLSIFCFYSRAFEKMFQQCLELPSQSRYSIAFPLLCTHFMSCTHELCPEERHHIGDRSLSLCNMFLDEMAKQARNLITDICTEQCTLSDQLLPKHCAKTISQAVNKKSKKQTGKKGEPEREKPGVESMRKNRLVVTNLDKLHTALSELCFSINYVPNMVVWEHTFTPREYLTSHLEIRFTKSIVGMTMYNQATQEIAKPSELLTSVRAYMTVLQSIENYVQIDITRVFNNVLLQQTQHLDSHGEPTITSLYTNWYLETLLRQVSNGHIAYFPAMKAFVNLPTENELTFNAEEYSDISEMRSLSELLGPYGMKFLSESLMWHISSQVAELKKLVVENVDVLTQMRTSFDKPDQMAALFKRLSSVDSVLKRMTIIGVILSFRSLAQEALRDVLSYHIPFLVSSIEDFKDHIPRETDMKVAMNVYELSSAAGLPCEIDPALVVALSSQKSENISPEEEYKIACLLMVFVAVSLPTLASNVMSQYSPAIEGHCNNIHCLAKAINQIAAALFTIHKGSIEDRLKEFLALASSSLLKIGQETDKTTTRNRESVYLLLDMIVQESPFLTMDLLESCFPYVLLRNAYHAVYKQSVTSSA

SEQ ID NO: 7 - protein, 211 aa, human
MANAGLQLLGFILAFLGWIGAIVSTALPQWRIYSYAGDNIVTAQAMYEGLWMSCVSQSTGQIQCKVFDSLLNLSSTLQATRALMVVGILLGVIAIFVATVGMKCMKCLEDDEVQKMRMAVIGGAIFLLAGLAILVATAWYGNRIVQEFYDPMTPVNARYEFGQALFTGWAAASLCLLGGALLCCSCPRKTTSYPTPRPYPKPAPSSGKDYV

SEQ ID NO: 8 - protein, 220 aa, human
MSMGLEITGTALAVLGWLGTIVCCALPMWRVSAFIGSNIITSQNIWEGLWMNCVVQSTGQMQCKVYDSLLALPQDLQAARALIVVAILLAAFGLLVALVGAQCTNCVQDDTAKAKITIVAGVLFLLAALLTLVPVSWSANTIIRDFYNPVVPEAQKREMGAGLYVGWAAAALQLLGGALLCCSCPPREKKYTATKVVYSAPRSTGPGASLGTGYDRKDYV

SEQ ID NO: 9 - protein, 135 aa, human
MATVQQLEGRWRLVDSKGFDEYMKELGVGIALRKMGAMAKPDCIITCDGKNLTIKTESTLKTTQFSCTLGEKFEETTADGRKTQTVCNFTDGALVQHQEWDGKESTITRKLKDGKLVVECVMNNVTCTRIYEKVE

SEQ ID NO: 10 - protein, 522 aa, human
MSSRPLESPPPYRPDEFKPNHYAPSNDIYGGEMHVRPMLSQPAYSFYPEDEILHFYKWTSPPGVIRILSMLIIVMCIAIFACVASTLAWDRGYGTSLLGGSVGYPYGGSGFGSYGSGYGYGYGYGYGYGGYTDPRAAKGFMLAMAAFCFIAALVIFVTSVIRSEMSRTRRYYLSVIIVSAILGIMVFIATIVYIMGVNPTAQSSGSLYGSQIYALCNQFYTPAATGLYVDQYLYHYCVVDPQEAIAIVLGFMIIVAFALIIFFAVKTRRKMDRYDKSNILWDKEHIYDEQPPNVEEWVKNVSAGTQDVPSPPSDYVERVDSPMAYSSNGKVNDKRFYPESSYKSTPVPEVVQELPLTSPVDDFRQPRYSSGGNFETPSKRAPAKGRAGRSKRTEQDHYETDYTTGGESCDELEEDWIREYPPITSDQQRQLYKRNFDTGLQEYKSLQSELDEINKELSRLDKELDDYREESEEYMAAADEYNRLKQVKGSADYKSKKNHCKQLKSKLSHIKKMVGDYDRQKT

SEQ ID NO: 11 - protein, 352 aa, human
MRSLGALLLLLSACLAVSAGPVPTPPDNIQVQENFNISRIYGKWYNLAIGSTCPWLKKIMDRMTVSTLVLGEGATEAEISMTSTRWRKGVCEETSGAYEKTDTDGKFLYHKSKWNITMESYVVHTNYDEYAIFLTKKFSRHHGPTITAKLYGRAPQLRETLLQDFRVVAQGVGIPEDSIFTMADRGECVPGEQEPEPILIPRVRRAVLPQEEEGSGGGQLVTEVTKKEDSCQLGYSAGPCMGMTSRYFYNGTSMACETFQYGGCMGNGNNFVTEKECLQTCRTVAACNLPIVRGPCRAFIQLWAFDAVKGKCVLFPYGGCQGNGNKFYSEKECREYCGVPGDGDEELLRFSN

SEQ ID NO: 12 - protein, 115 aa, human
MNAFLLSALCLLGAWAALAGGVTVQDGNFSFSLESVKKLKDLQEPQEPRVGKLRNFAPIPGEPVVPILCSNPNFPEELKPLCKEPNAQEILQRLEEIAEDPGTCEICAYAACTGC

SEQ ID NO: 13 - protein, 158 aa, human
MMQKLLKCSRLVLALALILVLESSVQGYPTQRARYQWVRCNPDSNSANCLEEKGPMFELLPGESNKIPRLRTDLFPKTRIQDLNRIFPLSEDYSGSGFGSGSGSGSGSGSGFLTEMEQDYQLVDESDAFHDNLRSLDRNLPSDSQDLGQHGLEEDFML

SEQ ID NO: 14 - protein, 17 aa, human
NLPSDSQDLGQHGLEED

SEQ ID NO: 15 - protein, 12 aa, human
VSAQQVQGVHAR

SEQ ID NO: 16 - protein, 16 aa, human
VTVQDGNFSFSLESVK

SEQ ID NO: 17 - protein, 12 aa, human
TGEINITSIVDR

SEQ ID NO: 18 - protein, 9 aa, human
YGDGIQLTR

SEQ ID NO: 19 - protein, 12 aa, human
VNSQSLSPYLFR

SEQ ID NO: 20 - protein, 22 aa, human
ILLMDLNEEDPTVLELGITGSK

SEQ ID NO: 21 - protein, 19 aa, human
AFPALTSLDLSDNPGLGER

SEQ ID NO: 22 - protein, 11 aa, human
SENISPEEEYK

SEQ ID NO: 23 - protein, 16 aa, human
VFDSLLNLSSTLQATR

SEQ ID NO: 24 - protein, 12 aa, human
DFYNPVVPEAQK

SEQ ID NO: 25 - protein, 9 aa, human
ELGVGIALR

SEQ ID NO: 26 - protein, 10 aa, human
NFDTGLQEYK

SEQ ID NO: 27 - protein, 9 aa, human
HHGPTITAK

(19) United States
(12) Patent Application Publication
(10) Pub. No.: US 2019/0383831 A1
(43) Pub. Date: Dec. 19, 2019

(54) DIAGNOSTIC METHOD

(71) Applicants: NEWSOUTH INNOVATIONS PTY LIMITED, Sydney (AU); SYDNEY LOCAL HEALTH DISTRICT, Camperdown (AU)

(72) Inventors: Valerie Christine WASINGER, Lugarno (AU); Rupert WING-LOONG LEONG, Glebe (AU); Yunki Yung YAU, Killara (AU); Sonia BUSTAMANTE, Mortdale (AU)

(21) Appl. No.: 16/308,360

(22) PCT Filed: Jun. 9, 2017
(86) PCT No.: PCT/AU2017/050580

§ 371 Date: Dec. 7, 2018

(30) Foreign Application Priority Data
Jun. 10, 2016 (AU) 2016902281

(51) Int. Cl. G01N 33/68; G01N 30/72

(52) U.S. Cl. CPC G01N 33/6893; G01N 2030/8813; G01N 30/72

(57) ABSTRACT

The invention relates to a method of diagnosing, assessing, and monitoring impaired gastrointestinal barrier function in a subject, comprising comparing the level of one or more markers in a tissue or body fluid of the subject relative to a reference value for the one or more markers, wherein the one or more markers are selected from the group consisting of: desmoglein 1 or a fragment thereof; desmoplakin or a fragment thereof; SPP 24 or a fragment thereof; Paraoxonase or a fragment thereof; CD14 protein precursor or a fragment thereof; Nck-associated protein 1 or a fragment thereof; Claudin-1 or a fragment thereof; Claudin-3 or a fragment thereof; Fatty acid-binding protein 5 or a fragment thereof; Occludin or a fragment thereof; Alpha-1-microglobulin or a fragment thereof; Guanylate cyclase activator 2a or a fragment thereof; Serglycin or a fragment thereof; Propanoic acid; Hydroxybutyric acid; Citric acid; Proline; Valine; 4-hydroxy-benzoic acid; and Anthranilic acid, and to compositions, devices and kits comprising the one or more markers for diagnosing, assessing, and monitoring impaired gastrointestinal barrier function in a subject.

### FIELD OF THE INVENTION

The invention relates to a method for diagnosing, assessing or monitoring impaired gastrointestinal barrier function in a subject, to methods of treating a subject diagnosed as suffering from impaired gastrointestinal barrier function, and to compositions and kits for diagnosing, assessing, or monitoring impaired gastrointestinal barrier function in a subject.

### BACKGROUND

Impaired gastrointestinal barrier function can result in failure of the gastrointestinal mucosal lining to maintain a barrier between gastrointestinal contents and the systemic circulation. As a consequence, gastrointestinal contents, such as intestinal lumenal microorganisms and parts thereof, microbial products, and other intestinal lumenal substances, can leak through the gastrointestinal lining into the systemic circulation. Leakage out of the gastrointestinal tract into the system circulation, known as “leaky gut syndrome”, can cause inflammation of the gastrointestinal tract and may underlie the pathogenesis of Crohn's Disease and Ulcerative Colitis.

Impaired gastrointestinal barrier function, such as leaky gut syndrome, is associated with approximately half of subjects already suffering from Crohn's disease and Ulcerative colitis patients. Leaky gut syndrome may be associated with complications of IBD (Inflammatory Bowel Disease) in subjects already suffering from IBD, such as the development of fistulas, strictures and dysplastic transformation in Crohn's disease and Ulcerative colitis.

One example of impaired gastrointestinal barrier function is complicated Crohn's disease. Half of all subjects suffering from Crohn's disease will experience a stricturing (SCD) or fistulizing (FCD) complication within 10 years from diagnosis. The cause of progression to complicated (SCD and FCD) disease (CCD) is unknown.

There is no one test that can reliably diagnose impaired gastrointestinal barrier function. Tests that are used for diagnosis of impaired gastrointestinal barrier function, such as leaky gut syndrome, include: conducting a colonoscopy to detect resolution of inflammation and mucosal healing or lack thereof; conducting colonoscopy to detect dye leakage into the gastrointestinal tract following intravenous injection of the dye; measurement of macromolecules, or ratios of macromolecules, in urinary excretion which may be altered due to increased intestinal absorption, trans-epithelial electrical resistance of bowel specimens; urinary chromium ADTA test. However, such tests are not definitive. For example, absence of detection of inflammation by colonoscopy does not necessarily indicate that impairment to the barrier function has been repaired. In this regard, repair of mucosal surfaces may occur prior to repair of the barrier function. Thus, failure to accurately predict impaired gastrointestinal barrier function can lead to insufficient treatment of the patient and subsequent prolonged suffering.

Moreover, in cases such as complicated CD, irreversible and cumulative damage may have occurred and the ensuing surgeries, prolonged hospitalizations and disability make up a significant component of the overall disease burden of CD.

The ability to accurately diagnose impaired gastrointestinal barrier function, such as leaky gut and CCD, would therefore allow better prediction of full repair of the bowel and the risk of further damage to the bowel may be reduced. Improved diagnosis of impaired gastrointestinal barrier function would allow flares of IBD to be predicted more readily so that treatment could be escalated at the appropriate time.

What is needed is a convenient and reliable method for the diagnosis of impaired gastrointestinal mucosal barrier function.

### SUMMARY

The inventors have identified markers in body fluid which are associated with the occurrence of, and/or the severity of, impaired gastrointestinal barrier function, such as leaky gut syndrome and CCD. Such markers can be used to diagnose, assess, or monitor impaired gastrointestinal barrier function.

A first aspect provides a method of diagnosing, assessing, or monitoring impaired gastrointestinal barrier function in a subject, comprising comparing the level of one or more markers in a tissue or body fluid of the subject relative to a reference value for the one or more markers, wherein the one or more markers are selected from the group consisting of:

i) desmoglein or a fragment thereof;

ii) desmoplakin or a fragment thereof;

iii) SPP 24 or a fragment thereof;

iv) Paraoxonase or a fragment thereof;

v) CD14 protein precursor or a fragment thereof;

vi) Nck-associated protein 1 or a fragment thereof;

vii) Claudin-1 or a fragment thereof;

viii) Claudin-3 or a fragment thereof;

ix) Fatty acid-binding protein 5 or a fragment thereof;

x) Occludin or a fragment thereof;

xi) Alpha-1-microglobulin or a fragment thereof;

xii) Guanylate cyclase activator 2a or a fragment thereof;

xiii) Serglycin or a fragment thereof;

xiv) Propanoic acid;

xv) Hydroxybutyric acid;

xvi) Citric acid;

xvii) Proline;

xviii) Valine;

xix) 4-hydroxy-benzoic acid; and

xx) Anthranilic acid.

A second aspect provides a method of diagnosing, assessing, or monitoring impaired gastrointestinal barrier function in a subject, comprising comparing the level of one or more markers in a tissue or body fluid of the subject relative to a reference value for the one or more markers, wherein the one or more markers are selected from the group consisting of:

i) desmoglein or a fragment thereof;

ii) SPP 24 or a fragment thereof;

iii) desmoplakin or a fragment thereof;

iv) Nck-associated protein 1 or a fragment thereof;

v) CD14 protein precursor or a fragment thereof;

vi) Fatty acid-binding protein or a fragment thereof;

vii) Claudin-1 or a fragment thereof;

viii) Claudin-3 or a fragment thereof;

ix) Occludin or a fragment thereof;

x) Guanylate cyclase activator 2a or a fragment thereof;

xi) Serglycin or a fragment thereof;

xii) Propanoic acid;

xiii) Hydroxybutyric acid;

xiv) Citric acid;

xv) Proline;

xvi) Valine;

xvii) 4-hydroxy-benzoic acid; and

xviii) Anthranilic acid,

wherein the reference value for the one or more markers is the level of the one or more markers in the tissue or body fluid of a subject not suffering from impaired gastrointestinal barrier function, and wherein impaired gastrointestinal barrier function is diagnosed when the level of the one or more markers is elevated relative to the reference value.

A third aspect provides a method of diagnosing, assessing, or monitoring leaky gut syndrome in a subject, comprising comparing the level of one or more markers in a tissue or body fluid of the subject relative to a reference value for the one or more markers, wherein the one or more markers are selected from the group consisting of:

(i) desmoglein or a fragment thereof;

(ii) SPP 24 or a fragment thereof;

(iii) desmoplakin or a fragment thereof;

(iv) Nck-associated protein 1 or a fragment thereof;

(v) CD14 protein precursor or a fragment thereof;

(vi) Fatty acid-binding protein or a fragment thereof;

(vii) Claudin-1 or a fragment thereof;

(viii) Claudin-3 or a fragment thereof;

(ix) Occludin or a fragment thereof;

(x) Guanylate cyclase activator 2a or a fragment thereof;

(xi) Serglycin or a fragment thereof;

(xii) Propanoic acid;

(xiii) Hydroxybutyric acid;

(xiv) Citric acid;

(xv) Proline;

(xvi) Valine;

(xvii) 4-hydroxy-benzoic acid; and

(xviii) Anthranilic acid,

wherein the reference value for the one or more markers is the level of the one or more markers in the tissue or body fluid of a subject not suffering from leaky gut syndrome, and wherein leaky gut syndrome is diagnosed when the level of the one or more markers is elevated relative to the reference value.

A fourth aspect provides a method of diagnosing, assessing, or monitoring leaky gut syndrome in a subject, comprising comparing the level of one or more markers in a tissue or body fluid of the subject relative to a reference value for the one or more markers, wherein the one or more markers are selected from the group consisting of:

(i) desmoglein or a fragment thereof;

(ii) SPP 24 or a fragment thereof;

(iii) Nck-associated protein 1 or a fragment thereof;

(iv) Claudin-1 or a fragment thereof;

(v) Claudin-3 or a fragment thereof;

(vi) Occludin or a fragment thereof;

(vii) Guanylate cyclase activator 2a or a fragment thereof; and

(viii) Serglycin or a fragment thereof,

wherein the reference value for the one or more markers is the level of the one or more markers in the tissue or body fluid of a subject suffering from leaky gut with low leak, and wherein leaky gut with high leak is diagnosed when the level of the one or more markers is elevated relative to the reference value.

A fifth aspect provides a method of diagnosing, assessing, or monitoring leaky gut syndrome in a subject, comprising comparing the level of one or more markers in a tissue or body fluid of the subject relative to a reference value for the one or more markers, wherein the one or more markers are selected from the group consisting of:

(i) Paraoxonase or a fragment thereof; and

(ii) Alpha-1-microglobulin or a fragment thereof,

wherein the reference value for the one or more markers is the level of the one or more markers in the tissue or body fluid of a subject not suffering from leaky gut syndrome, or suffering from leaky gut with low leak, and leaky gut syndrome with high leak is diagnosed when the level of the one or more markers is reduced relative to the reference value.

A sixth aspect provides a method of diagnosing, assessing or monitoring complicated Crohn's Disease (CCD) in a subject, comprising comparing the level of one or more markers in a tissue or body fluid of the subject relative to a reference value for the one or more markers, wherein the one or more markers are selected from the group consisting of:

(i) desmoglein or a fragment thereof;

(ii) desmoplakin or a fragment thereof;

(iii) SPP 24 or a fragment thereof;

(iv) CD14 protein precursor or a fragment thereof;

(v) Fatty acid-binding protein 5 or a fragment thereof;

(vi) Claudin-3 or a fragment thereof; and

(vii) Occludin or a fragment thereof,

wherein the reference value for the one or more markers is the level of the one or more markers in the tissue or body fluid of a subject not suffering from CCD, and wherein CCD is diagnosed when the level of the one or more markers is elevated relative to the reference value.

A seventh aspect provides a method of diagnosing or assessing complicated Crohn's disease (CCD) in a subject, comprising comparing the level of one or more markers in a tissue or body fluid of the subject relative to a reference value for the one or more markers, wherein the one or more markers are selected from the group consisting of:

(i) desmoglein or a fragment thereof;

(ii) desmoplakin or a fragment thereof; and

(iii) fatty acid-binding protein or a fragment thereof.

An eighth aspect provides a method of diagnosing or assessing complicated Crohn's disease (CCD) in a subject, comprising comparing the level of one or more markers in a tissue or body fluid of the subject relative to a reference value for the one or more markers, wherein the one or more markers are selected from the group consisting of:

(i) desmoglein or a fragment thereof;

(ii) desmoplakin or a fragment thereof; and

(iii) Fatty acid-binding protein or a fragment thereof,

wherein the reference value for the one or more markers is the level of the one or more markers in a subject not suffering from CCD, and wherein CCD is diagnosed when the level of the one or more markers is elevated relative to the reference value.

A ninth aspect provides use of one or more markers selected from the group consisting of:

i) desmoglein or a fragment thereof;

ii) desmoplakin or a fragment thereof;

iii) SPP 24 or a fragment thereof;

iv) Paraoxonase or a fragment thereof;

v) CD14 protein precursor or a fragment thereof;

vi) Nck-associated protein 1 or a fragment thereof;

vii) Claudin-1 or a fragment thereof;

viii) Claudin-3 or a fragment thereof;

ix) Fatty acid-binding protein or a fragment thereof;

x) Occludin or a fragment thereof;

xi) Alpha-1-microglobulin or a fragment thereof;

xii) Guanylate cyclase activator 2a or a fragment thereof;

xiii) Serglycin or a fragment thereof,

xiv) Propanoic acid;

xv) Hydroxybutyric acid;

xvi) Citric acid;

xvii) Proline;

xviii) Valine;

xix) 4-hydroxy-benzoic acid; and

xx) Anthranilic acid,

for diagnosing or assessing or monitoring impaired gastrointestinal barrier function in a subject.

A tenth aspect provides one or more markers selected from the group consisting of:

i) desmoglein or a fragment thereof;

ii) desmoplakin or a fragment thereof;

iii) SPP 24 or a fragment thereof;

iv) Paraoxonase or a fragment thereof;

v) CD14 protein precursor or a fragment thereof;

vi) Nck-associated protein 1 or a fragment thereof;

vii) Claudin-1 or a fragment thereof;

viii) Claudin-3 or a fragment thereof;

ix) Fatty acid-binding protein or a fragment thereof;

x) Occludin or a fragment thereof;

xi) Alpha-1-microglobulin or a fragment thereof;

xii) Guanylate cyclase activator 2a or a fragment thereof;

xiii) Serglycin or a fragment thereof;

xiv) Propanoic acid;

xv) Hydroxybutyric acid;

xvi) Citric acid;

xvii) Proline;

xviii) Valine;

xix) 4-hydroxy-benzoic acid; and

xx) Anthranilic acid,

for use in diagnosing or assessing or monitoring impaired gastrointestinal barrier function in a subject.

An eleventh aspect provides a composition for diagnosing or assessing or monitoring impaired gastrointestinal barrier function in a subject, comprising:
(a) one or more markers selected from the group consisting of:

(i) desmoglein or a fragment thereof;

(ii) desmoplakin or a fragment thereof;

(iii) SPP 24 or a fragment thereof;

(iv) Paraoxonase or a fragment thereof;

(v) CD14 protein precursor or a fragment thereof;

(vi) Nck-associated protein 1 or a fragment thereof;

(vii) Claudin-1 or a fragment thereof;

(viii) Claudin-3 or a fragment thereof;

(ix) Fatty acid-binding protein or a fragment thereof;

(x) Occludin or a fragment thereof;

(xi) Alpha-1-microglobulin or a fragment thereof;

(xii) Guanylate cyclase activator 2a or a fragment thereof;

(xiii) Serglycin or a fragment thereof;

(xiv) Propanoic acid;

(xv) Hydroxybutyric acid;

(xvi) Citric acid;

(xvii) Proline;

(xviii) Valine;

(xix) 4-hydroxy-benzoic acid; and

(xx) Anthranilic acid; or
(b) one or more antibodies or antigen binding fragments thereof, wherein each antibody or antigen binding fragment thereof specifically binds a marker selected from the group consisting of:
   (i) desmoglein or a fragment thereof;
   (ii) desmoplakin or a fragment thereof;
   (iii) SPP 24 or a fragment thereof;
   (iv) Paraoxonase or a fragment thereof;
   (v) CD14 protein precursor or a fragment thereof;
   (vi) Nck-associated protein 1 or a fragment thereof;
   (vii) Claudin-1 or a fragment thereof;
   (viii) Claudin-3 or a fragment thereof;
   (ix) Fatty acid-binding protein or a fragment thereof;
   (x) Occludin or a fragment thereof;
   (xi) Alpha-1-microglobulin or a fragment thereof;
   (xii) Guanylate cyclase activator 2a or a fragment thereof; and
   (xiii) Serglycin or a fragment thereof.

A twelfth aspect provides a device for diagnosing or assessing impaired gastrointestinal barrier function in a subject, comprising:
(a) one or more markers selected from the group consisting of:

(i) desmoglein or a fragment thereof;

(ii) desmoplakin or a fragment thereof;

(iii) SPP 24 or a fragment thereof;

(iv) Paraoxonase or a fragment thereof;

(v) CD14 protein precursor or a fragment thereof;

(vi) Nck-associated protein 1 or a fragment thereof;

(vii) Claudin-1 or a fragment thereof;

(viii) Claudin-3 or a fragment thereof;

(ix) Fatty acid-binding protein or a fragment thereof;

(x) Occludin or a fragment thereof;

(xi) Alpha-1-microglobulin or a fragment thereof;

(xii) Guanylate cyclase activator 2a or a fragment thereof;

(xiii) Serglycin or a fragment thereof;

(xiv) Propanoic acid;

(xv) Hydroxybutyric acid;

(xvi) Citric acid;

(xvii) Proline;

(xviii) Valine;

(xix) 4-hydroxy-benzoic acid; and

(xx) Anthranilic acid; or
(b) one or more antibodies or antigen binding fragments thereof, wherein each antibody or antigen binding fragment thereof specifically binds a marker selected from the group consisting of:
   (i) desmoglein or a fragment thereof;
   (ii) desmoplakin or a fragment thereof;
   (iii) SPP 24 or a fragment thereof;
   (iv) Paraoxonase or a fragment thereof;
   (v) CD14 protein precursor or a fragment thereof;
   (vi) Nck-associated protein 1 or a fragment thereof;
   (vii) Claudin-1 or a fragment thereof;
   (viii) Claudin-3 or a fragment thereof;
   (ix) Fatty acid-binding protein or a fragment thereof;
   (x) Occludin or a fragment thereof;
   (xi) Alpha-1-microglobulin or a fragment thereof;
   (xii) Guanylate cyclase activator 2a or a fragment thereof; and
   (xiii) Serglycin or a fragment thereof.

A thirteenth aspect provides a kit when used for diagnosing or assessing impaired gastrointestinal barrier function in a subject, comprising:
(a) one or more markers selected from the group consisting of:

(i) desmoglein or a fragment thereof;

(ii) desmoplakin or a fragment thereof;

(iii) SPP 24 or a fragment thereof;

(iv) Paraoxonase or a fragment thereof;

(v) CD14 protein precursor or a fragment thereof;

(vi) Nck-associated protein 1 or a fragment thereof;

(vii) Claudin-1 or a fragment thereof;

(viii) Claudin-3 or a fragment thereof;

(ix) Fatty acid-binding protein or a fragment thereof;

(x) Occludin or a fragment thereof;

(xi) Alpha-1-microglobulin or a fragment thereof;

(xii) Guanylate cyclase activator 2a or a fragment thereof;

(xiii) Serglycin or a fragment thereof;

(xiv) Propanoic acid;

(xv) Hydroxybutyric acid;

(xvi) Citric acid;

(xvii) Proline;

(xviii) Valine;

(xix) 4-hydroxy-benzoic acid; and

(xx) Anthranilic acid; or
(b) one or more antibodies or antigen binding fragments thereof, wherein each antibody or antigen binding fragment thereof specifically binds a marker selected from the group consisting of:
   (i) desmoglein or a fragment thereof;
   (ii) desmoplakin or a fragment thereof;
   (iii) SPP 24 or a fragment thereof;
   (iv) Paraoxonase or a fragment thereof;
   (v) CD14 protein precursor or a fragment thereof;
   (vi) Nck-associated protein 1 or a fragment thereof;
   (vii) Claudin-1 or a fragment thereof;
   (viii) Claudin-3 or a fragment thereof;
   (ix) Fatty acid-binding protein or a fragment thereof;
   (x) Occludin or a fragment thereof;
   (xi) Alpha-1-microglobulin or a fragment thereof;
   (xii) Guanylate cyclase activator 2a or a fragment thereof; and
   (xiii) Serglycin or a fragment thereof.

A fourteenth aspect provides a method of treating a subject diagnosed as suffering from impaired gastrointestinal barrier function, comprising administering an effective amount of an anti-inflammatory agent, an immune modifier, or an anti-TNF agent, wherein tissue or body fluid of the subject has been determined to contain elevated levels of one or more markers relative to a reference value for the one or more markers, wherein the reference value for the one or more markers is the level of the one or more markers in the tissue or body fluid of a subject not suffering from impaired gastrointestinal barrier function, and wherein the one or more markers are selected from the group consisting of:

i) desmoglein or a fragment thereof;

ii) SPP 24 or a fragment thereof;

iii) desmoplakin or a fragment thereof;

iv) Nck-associated protein 1 or a fragment thereof;

v) CD14 protein precursor or a fragment thereof;

vi) Fatty acid-binding protein or a fragment thereof;

vii) Claudin-1 or a fragment thereof;

viii) Claudin-3 or a fragment thereof;

ix) Occludin or a fragment thereof;

x) Guanylate cyclase activator 2a or a fragment thereof;

xi) Serglycin or a fragment thereof;

xii) Propanoic acid;

xiii) Hydroxybutyric acid;

xiv) Citric acid;

xv) Proline;

xvi) Valine;

xvii) 4-hydroxy-benzoic acid; and

xviii) Anthranilic acid.

A fifteenth aspect provides a method of treating a subject diagnosed as suffering from leaky gut syndrome, comprising administering an effective amount of an anti-inflammatory agent, an immune modifier, or an anti-TNF agent, wherein tissue or body fluid of the subject has been determined to contain elevated levels of one or more markers relative to a reference value for the one or more markers, wherein the reference value for the one or more markers is the level of the one or more markers in the tissue or body fluid of a subject not suffering from leaky gut syndrome, and wherein the one or more markers are selected from the group consisting of:

i) desmoglein or a fragment thereof;

ii) SPP 24 or a fragment thereof;

iii) desmoplakin or a fragment thereof;

iv) Nck-associated protein 1 or a fragment thereof;

v) CD14 protein precursor or a fragment thereof;

vi) Fatty acid-binding protein or a fragment thereof;

vii) Claudin-1 or a fragment thereof;

viii) Claudin-3 or a fragment thereof;

ix) Occludin or a fragment thereof;

x) Guanylate cyclase activator 2a or a fragment thereof;

xi) Serglycin or a fragment thereof;

xii) Propanoic acid;

xiii) Hydroxybutyric acid;

xiv) Citric acid;

xv) Proline;

xvi) Valine;

xvii) 4-hydroxy-benzoic acid; and

xviii) Anthranilic acid.

A sixteenth aspect provides a method of treating a subject diagnosed as suffering from CCD, comprising administering an effective amount of an anti-inflammatory agent, an immune modifier, or an anti-TNF agent, wherein tissue or body fluid of the subject has been determined to contain elevated levels of one or more markers relative to a reference value for the one or more markers, wherein the reference value for the one or more markers is the level of the one or more markers in tissue or body fluid of a subject not suffering from CCD, and wherein the one or more markers are selected from the group consisting of:

i) desmoglein or a fragment thereof;

ii) desmoplakin or a fragment thereof; and

iii) Fatty acid-binding protein or a fragment thereof.

A seventeenth aspect provides a method of diagnosing, assessing, or monitoring leaky gut syndrome in a subject, comprising determining the level of one or more markers in a tissue or body fluid of the subject relative to a reference value for the one or more markers, wherein the one or more markers are selected from the group consisting of:
(i) desmoglein or a fragment thereof;
(ii) SPP 24 or a fragment thereof;
(iii) Nck-associated protein 1 or a fragment thereof;
(iv) Claudin-1 or a fragment thereof;
(v) Claudin-3 or a fragment thereof;
(vi) Occludin or a fragment thereof;
(vii) Guanylate cyclase activator 2a or a fragment thereof; and
(viii) Serglycin or a fragment thereof,
   wherein the level of the one or more markers is determined in the tissue or body fluid with one or more antibodies or antigen binding fragments thereof that specifically binds to the one or more markers, wherein the reference value is the level of the one or more markers in a subject suffering from leaky gut with low leak, and wherein leaky gut with high leak is diagnosed when the level of the one or more markers is elevated relative to the reference value.

An eighteenth aspect provides a method of diagnosing, assessing, or monitoring leaky gut syndrome in a subject, comprising determining the level of one or more markers in a tissue or body fluid of the subject relative to a reference value for the one or more markers, wherein the one or more markers are selected from the group consisting of:

(iii) paraoxonase or a fragment thereof; and

(iv) alpha-1-microglobulin or a fragment thereof,

wherein the level of the one or more markers is determined in the tissue or body fluid with one or more antibodies or antigen binding fragments thereof that specifically binds to the one or more markers, wherein the reference value is the level of the one or more markers in a subject not suffering from leaky gut syndrome, or suffering from leaky gut with low leak, and wherein leaky gut syndrome with high leak is diagnosed when the level of the one or more markers is reduced relative to the reference value.

A nineteenth aspect provides a method of diagnosing, assessing, or monitoring complicated Crohn's disease (CCD) in a subject, comprising determining the level of one or more markers in a tissue or body fluid of the subject relative to a reference value for the one or more markers, wherein the one or more markers are selected from the group consisting of:

(i) desmoglein or a fragment thereof;

(ii) desmoplakin or a fragment thereof;

(iii) Fatty acid-binding protein or a fragment thereof;

(iv) SPP 24 or a fragment thereof;

(v) CD14 protein precursor or a fragment thereof;

(vi) Fatty acid-binding protein 5 or a fragment thereof;

(vii) Claudin-3 or a fragment thereof; and

(viii) Occludin or a fragment thereof,

wherein the level of the one or more markers is determined in the tissue or body fluid with one or more antibodies or antigen binding fragments thereof that specifically binds to the one or more markers, wherein the reference value is the level of the one or more markersin a subject not suffering from CCD, and wherein CCD is diagnosed when the level of the one or more markersis elevated relative to the reference value.

A twentieth aspect provides a method of diagnosing, assessing, or monitoring impaired gastrointestinal barrier function in a subject, comprising:
(a) contacting a tissue or body fluid sample from the subject with a proteolytic enzyme to produce a digested sample;
(b) performing mass spectrometry to determine the level of one or more markers in the digested sample, wherein the one or more markers are selected from the group consisting of:
   i) desmoglein or a fragment thereof;
   ii) desmoplakin or a fragment thereof;
   iii) SPP 24 or a fragment thereof;
   iv) Paraoxonase or a fragment thereof;
   v) CD14 protein precursor or a fragment thereof;
   vi) Nck-associated protein 1 or a fragment thereof;
   vii) Claudin-1 or a fragment thereof;
   viii) Claudin-3 or a fragment thereof;
   ix) Fatty acid-binding protein 5 or a fragment thereof;
   x) Occludin or a fragment thereof;
   xi) Alpha-1-microglobulin or a fragment thereof;
   xii) Guanylate cyclase activator 2a or a fragment thereof;
   xiii) Serglycin or a fragment thereof;
   xiv) Propanoic acid;
   xv) Hydroxybutyric acid;
   xvi) Citric acid;
   xvii) Proline;
   xviii) Valine;
   xix) 4-hydroxy-benzoic acid; and
   xx) Anthranilic acid; and
(c) comparing the level of the one or more markers in the sample relative to a reference value for the one or more markers.

A twentyfirst aspect provides a method of diagnosing, assessing, or monitoring impaired gastrointestinal barrier function in a subject, comprising:
(a) Contacting a tissue or body fluid sample from the subject with a proteolytic enzyme to produce a digested sample;
(b) performing mass spectrometry to determine the level of one or more markers in the digested sample, wherein the one or more markers are selected from the group consisting of:
   (i) desmoglein or a fragment thereof;
   (ii) SPP 24 or a fragment thereof;
   (iii) desmoplakin or a fragment thereof;
   (iv) Nck-associated protein 1 or a fragment thereof;
   (v) CD14 protein precursor or a fragment thereof;
   (vi) Fatty acid-binding protein or a fragment thereof;
   (vii) Claudin-1 or a fragment thereof;
   (viii) Claudin-3 or a fragment thereof;
   (ix) Occludin or a fragment thereof;
   (x) Guanylate cyclase activator 2a or a fragment thereof;
   (xi) Serglycin or a fragment thereof;
   (xii) Propanoic acid;
   (xiii) Hydroxybutyric acid;
   (xiv) Citric acid;
   (xv) Proline;
   (xvi) Valine;
   (xvii) 4-hydroxy-benzoic acid; and
   (xviii) Anthranilic acid,
(c) comparing the level of the one or more markers in the sample relative to a reference value for the one or more markers, wherein the reference value is the level of the one or more markers in tissue or body fluid of a subject not suffering from impaired gastrointestinal barrier function, and wherein impaired gastrointestinal barrier function is diagnosed when the level of the one or more markers is elevated relative to the reference value.

A twentysecond aspect provides a method of diagnosing, assessing, or monitoring leaky gut syndrome in a subject, comprising:
(a) contacting a tissue or body fluid sample from the subject with a proteolytic enzyme to produce a digested sample;
(b) performing mass spectrometry to determine the level of one or more markers in the digested sample, wherein the one or more markers are selected from the group consisting of:
   (i) Paraoxonase or a fragment thereof; and
   (ii) Alpha-1-microglobulin or a fragment thereof, and
(c) comparing the level of the one or more markers in the sample relative to a reference value for the one or more markers, wherein the reference value is the level of the one or more markers in tissue or body fluid of a subject not suffering from leaky gut syndrome, or suffering from leaky gut with low leak, and wherein leaky gut syndrome with high leak is diagnosed when the level of the one or more markers is reduced relative to the reference value.

A twentythird aspect provides a method of diagnosing, assessing, or monitoring CCD in a subject, comprising:
(a) contacting a tissue or body fluid sample from the subject with a proteolytic enzyme to produce a digested sample;
(b) performing mass spectrometry to determine the level of one or more markers in the digested sample, wherein the one or more markers are selected from the group consisting of:
   (i) desmoglein or a fragment thereof;
   (ii) desmoplakin or a fragment thereof;
   (iii) Fatty acid-binding protein or a fragment thereof;
   (iv) SPP 24 or a fragment thereof;
   (v) CD14 protein precursor or a fragment thereof;
   (vi) Fatty acid-binding protein 5 or a fragment thereof;
   (vii) Claudin-3 or a fragment thereof; and
   (viii) Occludin or a fragment thereof; and
(c) comparing the level of the one or more markers in the tissue or body fluid of the subject relative to a reference value for the one or more markers, wherein the reference value is the level of the one or more markers in a subject not suffering from CCD, and wherein CCD is diagnosed when the level of the one or more markers is elevated relative to the reference value.

A twentyfourth aspect provides a panel for diagnosing, assessing, or monitoring impaired gastrointestinal barrier function in a subject, wherein the panel comprises:
(a) one or more markers selected from the group consisting of:

i) desmoglein or a fragment thereof;

ii) desmoplakin or a fragment thereof;

iii) SPP 24 or a fragment thereof;

iv) Paraoxonase or a fragment thereof;

v) CD14 protein precursor or a fragment thereof;

vi) Nck-associated protein 1 or a fragment thereof;

vii) Claudin-1 or a fragment thereof;

viii) Claudin-3 or a fragment thereof;

ix) Fatty acid-binding protein 5 or a fragment thereof;

x) Occludin or a fragment thereof;

xi) Alpha-1-microglobulin or a fragment thereof;

xii) Guanylate cyclase activator 2a or a fragment thereof;

xiii) Serglycin or a fragment thereof;

xiv) Propanoic acid;

xv) Hydroxybutyric acid;

xvi) Citric acid;

xvii) Proline;

xviii) Valine;

xix) 4-hydroxy-benzoic acid; and

xx) Anthranilic acid; and/or
(b) one or more antibodies or antigen binding fragments thereof, wherein each antibody or antigen binding fragment thereof specifically binds a marker selected from the group consisting of:
   (i) desmoglein or a fragment thereof;
   (ii) desmoplakin or a fragment thereof;
   (iii) SPP 24 or a fragment thereof;
   (iv) Paraoxonase or a fragment thereof;
   (v) CD14 protein precursor or a fragment thereof;
   (vi) Nck-associated protein 1 or a fragment thereof;
   (vii) Claudin-1 or a fragment thereof;
   (viii) Claudin-3 or a fragment thereof;
   (ix) Fatty acid-binding protein or a fragment thereof;
   (x) Occludin or a fragment thereof;
   (xi) Alpha-1-microglobulin or a fragment thereof;
   (xii) Guanylate cyclase activator 2a or a fragment thereof; and
   (xiii) Serglycin or a fragment thereof.

A twentyfifth aspect provides a method of treating a subject diagnosed as suffering from leaky gut syndrome with high leak, comprising administering an effective amount of an anti-inflammatory agent, an immune modifier, or an anti-TNF agent, wherein tissue or body fluid of the subject has been determined to contain reduced levels of one or more markers relative to a reference value for the one or more markers, wherein the reference value for the one or more markers is the level of the one or more markers in tissue or body fluid of a subject not suffering from leaky gut syndrome, or suffering from leaky gut with low leak, and wherein the one or more markers are selected from the group consisting of:

(i) paraoxonase or a fragment thereof; and

(ii) alpha-1-microglobulin or a fragment thereof.

### BRIEF DESCRIPTION OF THE DRAWINGS

FIG. 1 shows the full length amino acid sequence of A. human desmoglein (SEQ ID NO: 1); B. human desmoplakin (SEQ ID NO: 2); C. human SPP 24 (SEQ ID NO: 3); D. human Paraoxonase (SEQ ID NO: 4); and E. human CD14 protein precursor (SEQ ID NO: 5); F. human Nck-associated protein 1 (SEQ ID NO: 6); G. human Claudin-1 (SEQ ID NO: 7); H. human claudin-3 (SEQ ID NO: 8); I. human Fatty acid-binding protein 5 (SEQ ID NO: 9); J. human Occludin (SEQ ID NO: 10); K. human Alpha-1-microglobulin (SEQ ID NO: 11); L. human Guanylate cyclase activator 2a (SEQ ID NO: 12); M. human Serglycin (SEQ ID NO: 13). Peptides within the full length sequences that have been detected following MRM of enzyme digested samples and/or ion count MS as described herein are underlined.

FIG. 2 is a graphs showing relative abundance of the peptide ILLMDLNEEDPTVLELGITGSK (SEQ ID NO: 20) from PON1 in subjects suffering from leaky gut with low and high leak, as determined by MRM analysis of trypsin digested serum samples.

FIG. 3 is a graph of the relative abundance of the proteins CD14, NCKP1 and SPP24 in pooled serum samples from subjects suffering from leaky gut with low and high leak, as determined by MRM analysis of trypsin digested serum samples.

FIG. 4 is a graph showing the ratio of the abundance of AMBP, GUC2a and SRGN in high leak subjects to the abundance of AMBP, GUC2a and SRGN in low leak subjects, as determined by MRM analysis of enzyme digested serum samples.

FIG. 5 is a graph showing the relative abundance of claudin 1 or claudin 3 in subjects suffering from leaky gut with high leak or low leak, as determined by MRM analysis of enzyme digested serum samples.

FIG. 6 is a graph showing the relative abundance of occludin in pooled samples from subjects suffering from leaky gut with low leak or high leak, as determined by MRM analysis of enzyme digested serum samples.

FIG. 7 is a graph showing the relative abundance of desmoglein (DSG1), desmoplakin (DESP) and fatty acid binding protein 5 (FABPS) in patients suffering from leaky gut with low leak or high leak, as determined by MRM analysis of enzyme digested serum samples.

FIG. 8 is a graph showing the ratio of the amount of desmoglein (DSG1), desmoplakin (DESP) and fatty acid binding protein 5 (FABPS) in serum of patients suffering from severe CD to the amount of desmoglein, desmoplakin and fatty acid binding protein 5 in serum of patients suffering from CD in remission.

FIG. 9 is a graph showing the emPAI values for various proteins bound to approximately 100 μg of the peptide VSAQQVQGVHAR immobilised on magnetic beads.

FIG. 10 is a graph showing that serum levels of desmoglein (DSG1), desmoplakin (DSK) and fatty acid binding protein 5 (FABPS) are increased in CCD by label-free LC-MS/MS. Relative A) DSK B) FABPS and C) DSG1 levels in CCD and ICD. D) A FABPS proteotypic peptide (ELGVGIALR) was sequenced by LC-MS/MS and contributed to FABP5 quantitative comparisons between ICD and CCD (FABP5 identified and quantified by 3 non-conflicting peptides).

FIG. 11 is a graph showing verification of epithelial-component biomarker candidates in Complicated Crohn's disease in a further cohort. A) Serum dot blots ordered by group. B) Normalized relative levels of DSG1, C) DSK and D) FABP5 between comparative groups. E) Total DSG1, DSK and FABP5 levels and the weighted F) SEC score between groups. One-way ANOVA P-values are stated where statistically significant (P<0.05).

FIG. 12 is a graph showing classification ability of serological epithelial component proteins for Complicated Crohn's disease. Total DSG1, DSK and FABP5 levels were able to classify: A) CD against RA, UC and control groups, B) CCD against ICD, RA, UC and controls, and C) CCD against ICD. The putative Serological Epithelial Component (SEC) score, developed using discriminant function coefficients of the biomarker candidates, improved classification of CD and CCD in all cases: D) CD against RA, UC and control groups, E) CCD against ICD, RA, UC and controls, and F) CCD against ICD.

FIG. 13 is a graph showing significant associations between biomarker candidates and clinical characteristics. A) Total DSG1, DSK and FABP5 levels were lower in subjects on mesalazines (3.11±0.23 vs. 1.95±0.29), and in subjects without B) previous resection(s) (3.36±0.29 vs. 2.40±0.23). C) Total DSG1, DSK and FABP5 levels were also moderately correlated with ESR (r=0.484). D) The SEC score remained lower in subjects on mesalazines (1.96±0.13 vs. 1.29±0.21) and was E) higher in subjects on biological therapies (2.07±0.11 vs. 1.58±0.14). F) SEC score also maintained a moderate correlation with ESR (r=0.492).

FIG. 14 is a graph showing FABP5 levels in CCD verified by NCI-CPTAC Tier-2 level MRM assay. A) Calibration curve for FABP5 MRM assay. A coefficient of determination of r2=0.99 was achieved using a polynomial regression equation (x=−18.475y2 +149.37y - 34.974) for a 1-300 pg/mL range of quantification. B) Quantitative performance of the FABP5 assay against a low-error criterion of zero back-calculated error±15%. The back-calculated error margin of the assay was 1.7% and precision % CV was 12.4. The inter-day full analytical replicate % CV was 10.7. C) Serum FABP5 levels were increased in CCD compared to all other groups. D) FABP5 levels were moderately correlated with age (rs=0.521), and E) age at diagnosis (rs=0.561). F) FABP5 levels were stable in active CCD subjects sampled at two time points between 54±14 days (221.45±55.33 vs. 200.13±52.84, p=0.829).

### DETAILED DESCRIPTION

The invention relates in one aspect to a method of diagnosing, assessing or monitoring impaired gastrointestinal barrier function in a subject. The impaired gastrointestinal barrier function is diagnosed, assessed or monitored by comparing the level of one or more markers in tissue or body fluid of the subject relative to a reference value for the one or more markers.

As used herein, “impaired gastrointestinal barrier function” refers to leaky gut syndrome and complications of IBD. Leaky gut syndrome (also referred to herein as “leaky gut”), occurs when the epithelial integrity of the gastrointestinal tract is compromised, allowing lumenal contents of the gut to enter the systemic circulation. This allows opportunistic organisms and their products to enter the blood stream. This “leak” can trigger an inflammatory response, which over time can be chronic. Leaky gut is believed to be one of the triggers for IBD, and may possibly be a trigger for other conditions such as heart disease, dementia, cancer, multiple sclerosis, chronic fatigue syndrome.

Leaky gut can occur when IBD is a pre-existing condition. In such circumstances, it is especially difficult to determine whether a subject is suffering from leaky gut.

The extent to which the gastrointestinal tract can leak can vary from subject to subject. In general, when the gastrointestinal barrier function is impaired, the leakiness of the gastrointestinal tract can be classified as low leak or high leak. High leak and low leak patients can be categorised based on confocal endomicrosopy leakiness score (CLE score) as described in Paramsothy, S., Leong, R. W. L. 2010. Fluorescein contrast in confocal laser endomicroscopy. Nature Reviews: Gastroenterology and Hepatology; Chang J, Ip M, Yang M, Wong B, Power T, Lin L, Xuan W, Phan T G, Leong R W.2016. The learning curve, interobserver, and intraobserver agreement of endoscopic confocal laser endomicroscopy in the assessment of mucosal barrier defects.Gastrointest Endosc. 83(4):785-791.

As used herein, “Inflammatory bowel disease” or “IBD” refers to Crohn's disease and Ulcerative colitis, collectively. Crohn's disease (CD) is an inflammation anywhere along the digestive tract, including the large intestine, and involves all layers of the intestinal wall. Common symptoms of CD include chronic diarrhea fever, abdominal pain, weight loss and lack of appetite. Ulcerative colitis (UC) is an inflammation of the large intestine and is associated with ulcers in the inner layers of the lining of the large intestine. Common symptoms of UC include bloody diarrhea, fever and abdominal pain.

Is some embodiments, leaky gut syndrome occurs in subjects suffering from IBD.

In some embodiments, leaky gut syndrome occurs in subjects that are not suffering from IBD.

In some embodiments, the impaired gastrointestinal barrier function is a complication of IBD, such as complicated CD (CCD). Complicated CD occurs in subjects already suffering from CD, and includes fistulas (fistulising CD) and/or strictures (stricturing CD) in the bowel, which typically extends through the submucosal layers. Thus, in one aspect, there is provided a method of diagnosing, assessing or monitoring CCD.

As used herein, a “marker” is a molecular indicator of a specific biological property or condition. A marker may be any chemical compound including, for example, a protein, a peptide, an organic acid (e.g. amino acid), etc.

The inventors have found that by using peptides of SPP24 or GUC2a immobilised on a solid support, such as magnetic beads, compounds which bind to the peptides of SPP24 or GUC2a can be isolated which are indicators of impaired gastrointestinal barrier function.

In some embodiments, the one or more markers comprises a compound which binds to SPP24 or a fragment thereof, or guanylin or a fragment thereof.

In one embodiment, the one or more markers comprise a compound which binds to the peptide VSAQQVQGVHAR (SEQ ID NO: 15) or

VTVQDGNFSFSLESVK SEQ ID NO: 16). In one embodiment, the one or more markers is one or more proteins which comprise a peptide which binds to the peptide VSAQQVQGVHAR or VTVQDGNFSFSLESVK. In one embodiment, the one or more markers is one or more peptides which bind to the peptide VSAQQVQGVHAR or VTVQDGNFSFSLESVK. In one embodiment, the one or more markers is one or more metabolites which bind to the peptide VSAQQVQGVHAR or VTVQDGNFSFSLESVK. The relative abundance of protein bound to the peptide may be expressed as the exponentially modified protein abundance index (emPAI), which is a method of estimating protein abundance from peptide counts in a single LC-MS/MS experiment. emPAI is defined as 10^{PAI} minus 1, where PAI (Protein Abundance Index) is the ratio of observed peptides to observable peptides. Methods for calculating emPAI are described in, for example, Ishihama et al. (2005) Molecular & Cellular Proteomics 4, 1265-1272. In one embodiment, the emPAI value for a marker described herein bound to 100 μg of the peptide VSAQQVQGVHAR is in the range of from 0.01 to 10, 0.02 to 10, 0.03 to 10, 0.03 to 7.

In one embodiment, the one or more markers are selected from the group consisting of:

i) desmoglein or a fragment thereof;

ii) desmoplakin or a fragment thereof;

iii) SPP 24 or a fragment thereof;

iv) Paraoxonase or a fragment thereof;

v) CD14 protein precursor or a fragment thereof;

vi) Nck-associated protein 1 or a fragment thereof;

vii) Claudin-1 or a fragment thereof;

viii) Claudin-3 or a fragment thereof;

ix) Fatty acid-binding protein 5 or a fragment thereof;

x) Occludin or a fragment thereof;

xi) Alpha-1-microglobulin or a fragment thereof;

xii) Guanylate cyclase activator 2a or a fragment thereof; and

xiii) Serglycin or a fragment thereof.

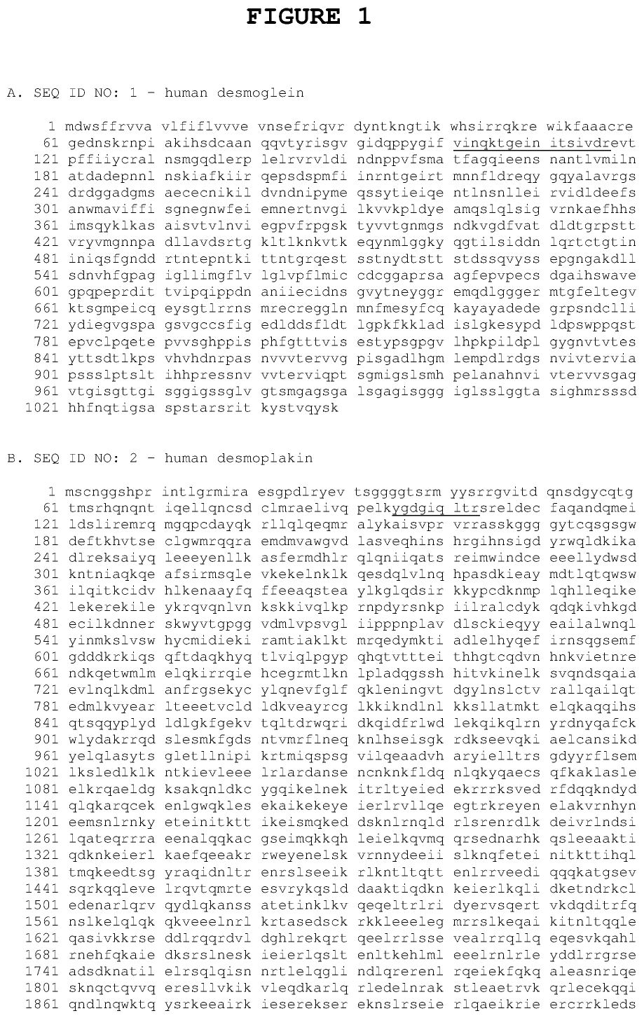

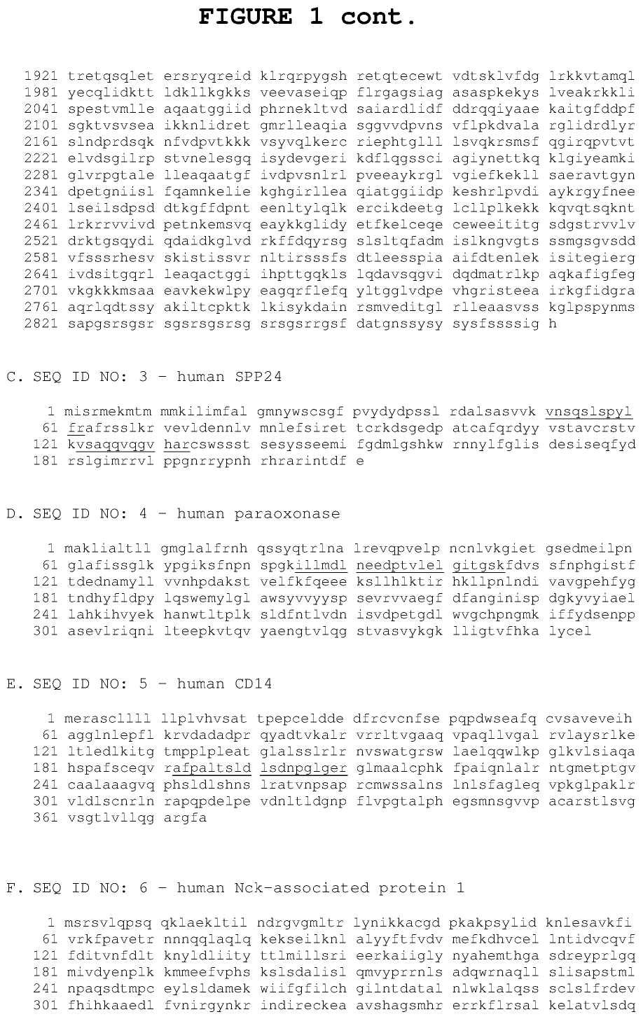

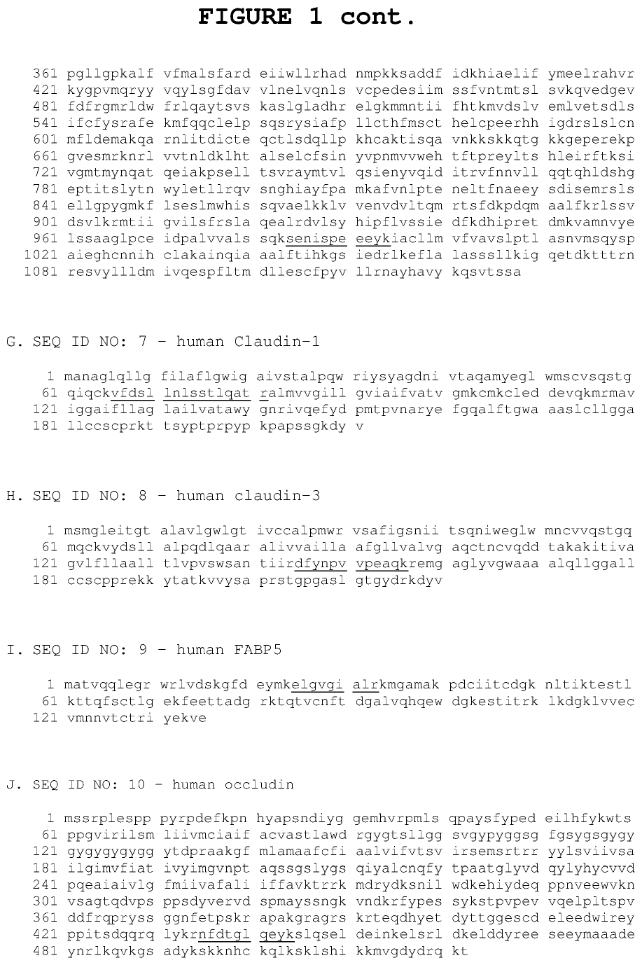

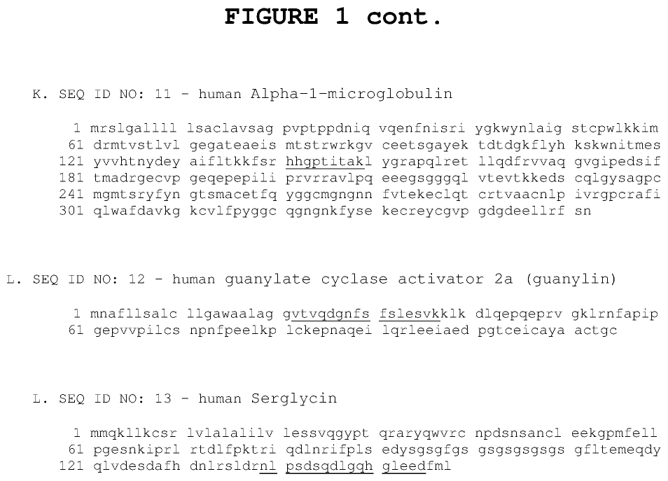

Desmoglein 1 (also referred to herein as DSG1 or desmoglein) is a cadherin which plays a role in the formation of desmosomes. The full length amino acid sequence of human desmoglein 1 is shown in FIG. 1 (SEQ ID NO: 1). In embodiments in which the one or more markers comprise desmoglein 1 or a fragment thereof, the desmoglein or fragment thereof may:
i) comprise an amino acid sequence of SEQ ID NO: 1 or fragment thereof; or
ii) comprise the amino acid sequence TGEINITSIVDR (SEQ ID NO: 17); or
iii) consist essentially of the amino acid sequence of SEQ ID NO: 17; or
iv) consist of the amino acid sequence of SEQ ID NO: 17.

Desmoplakin (also referred to herein as DESP or DSK)) is a protein which plays a role in the formation of desmosomes in cardiac muscle and epidermal cells. The full length amino acid sequence of human desmoplakin is shown in FIG. 1 (SEQ ID NO: 3). In embodiments in which the one or more markers comprise desmoplakin or a fragment thereof, the desmoplakin or fragment thereof may:
i) comprise an amino acid sequence of SEQ ID NO: 2 or fragment thereof; or
ii) comprise the amino acid sequence YGDGIQLTR (SEQ ID NO: 18); or
iii) consist essentially of the amino acid sequence of SEQ ID NO: 18; or
iv) consist of the amino acid sequence of SEQ ID NO: 18.

SPP24 is also known as secreted phosphoprotein 24 or secreted phosphoprotein 2. The full length amino acid sequence of human SPP24 is shown in FIG. 1 (SEQ ID NO: 3). In embodiments in which the one or more markers comprise SPP24 or a fragment thereof, the SPP24 or fragment thereof may:
i) comprise an amino acid sequence of SEQ ID NO: 3 or fragment thereof; or
ii) comprise an amino acid sequence VSAQQVQGVHAR (SEQ ID NO: 15); or
iii) consist essentially of the amino acid sequence of SEQ ID NO: 15; or
iv consist of the amino acid sequence of SEQ ID NO: 15; or
v) comprise an amino acid sequence VNSQSLSPYLFR (SEQ ID NO: 19); or
vi) consist essentially of the amino acid sequence of SEQ ID NO: 19; or
vii) consist of the amino acid sequence of SEQ ID NO: 19.

Paraoxonase (PON1) is a protein which prevents the oxidation of LDL. The full length amino acid sequence of human Paraoxonase is shown in FIG. 1 (SEQ ID NO: 4). In embodiments in which the one or more markers comprise Paraoxonase or a fragment thereof, the Paraoxonase or fragment thereof may:
i) comprise an amino acid sequence of SEQ ID NO: 4 or fragment thereof;
ii) comprise the amino acid sequence ILLMDLNEEDPTVLELGITGSK (SEQ ID NO: 20);
iii) consist essentially of the amino acid sequence of SEQ ID NO: 20; or
iv) consist of the amino acid sequence of SEQ ID NO: 20.

The full length amino acid sequence of human CD14 protein precursor (CD14) is shown in FIG. 1 (SEQ ID NO: 5). In embodiments in which the one or more markers comprise CD14 protein precursor or a fragment thereof, the CD14 protein precursor or fragment thereof may:
i) comprise an amino acid sequence of SEQ ID NO: 5 or fragment thereof;
ii)comprise the amino acid sequence AFPALTSLDLSDNPGLGER (SEQ ID NO: 21);
iii)consist essentially of the amino acid sequence of SEQ ID NO: 21; or
iv) consist of the amino acid sequence of SEQ ID NO: 21.

The full length amino acid sequence of human Nck-associated protein 1 (NCKP1) is shown in FIG. 1 (SEQ ID NO: 6). In embodiments in which the one or more markers comprise Nck-associated protein 1 or a fragment thereof, the Nck-associated protein 1 or fragment thereof may:
i)comprise an amino acid sequence of SEQ ID NO: 6 or fragment thereof;
ii) comprise the amino acid sequence SENISPEEEYK (SEQ ID NO: 22);
iii) consist essentially of the amino acid sequence of SEQ ID NO: 22; or
iv) consist of the amino acid sequence of SEQ ID NO: 22.

The full length amino acid sequence of human Claudin-1 (CLD1) is shown in FIG. 1 (SEQ ID NO: 7). In embodiments in which the one or more markers comprise Claudin-1 or a fragment thereof, the Claudin-1 or fragment thereof may:
i) comprise an amino acid sequence of SEQ ID NO: 7 or fragment thereof;
ii) comprise the amino acid sequence VFDSLLNLSSTLQATR (SEQ ID NO: 23);
iii) consist essentially of the amino acid sequence of SEQ ID NO: 23; or
iv) consist of the amino acid sequence of SEQ ID NO: 23.

The full length amino acid sequence of human Claudin-3 (CLD3) is shown in FIG. 1 (SEQ ID NO: 8). In embodiments in which the one or more markers comprise Claudin-3 or a fragment thereof, the Claudin-3 or fragment thereof may:
i) comprise an amino acid sequence of SEQ ID NO: 9 or fragment thereof;
ii) comprise the amino acid sequence DFYNPVVPEAQK (SEQ ID NO: 24);
iii) consist essentially of the amino acid sequence of SEQ ID NO: 24; or
iv) consist of the amino acid sequence of SEQ ID NO: 24.

The full length amino acid sequence of human fatty acid-binding protein 5 (FABP5) is shown in FIG. 1 (SEQ ID NO: 9). In embodiments in which the one or more markers comprise FABP5 or a fragment thereof, the FABP5 or fragment thereof may:
i) comprise an amino acid sequence of SEQ ID NO: 10 or fragment thereof;
ii) comprise the amino acid sequence ELGVGIALR (SEQ ID NO: 25);
iii) consist essentially of the amino acid sequence of SEQ ID NO: 25; or
iv) consist of the amino acid sequence of SEQ ID NO: 25.

The full length amino acid sequence of human occludin (OCLN) is shown in FIG. 1 (SEQ ID NO: 10). In embodiments in which the one or more markers comprise occludin or a fragment thereof, the occludin or fragment thereof may:
(i) comprise an amino acid sequence of SEQ ID NO: 9 or fragment thereof;
(ii) comprise the amino acid sequence NFDTGLQEYK (SEQ ID NO: 26);
(i) consist essentially of the amino acid sequence of SEQ ID NO: 26; or
(ii) consist of the amino acid sequence of SEQ ID NO: 26.

AMBP or its cleavage products are also known as Alpha-1 microglycoprotein, alpha-1 microglobulin, inter-alpha-trypsin light chain, bikunin, EDC-1, HI-30, uronic acid-rich protein, trystatin. The full length amino acid sequence of AMBP is shown in FIG. 1 (SEQ ID NO: 11). In embodiments in which the one or more markers comprise AMBP or a fragment thereof, the AMBP or fragment thereof may:
(i) comprise an amino acid sequence of SEQ ID NO: 11 or fragment thereof;
(ii) comprise an amino acid sequence HHGPTITAK (SEQ ID NO: 27);
(iii) consist essentially of the amino acid sequence of SEQ ID NO: 27; or
(iv) consist of the amino acid sequence of SEQ ID NO: 27.

Guanylin (GUC2a) is also known and referred to herein as Guanylate cyclase activator 2a, and Guanylate cyclase activator 2. The full length amino acid sequence of human guanylin is shown in FIG. 1 (SEQ ID NO: 12). In embodiments in which the one or more markers comprise Guanylin or a fragment thereof, the Guanylin or fragment thereof may:
i) comprise an amino acid sequence of SEQ ID NO: 12 or fragment thereof;
ii) comprise an amino acid sequence VTVQDGNFSFSLESVK (SEQ ID NO: 16);
iii) consist essentially of the amino acid sequence of SEQ ID NO: 16;
iv) consist of the amino acid sequence of SEQ ID NO: 16.

Serglycin (SRGN) is also known as proteoglycan 1 core protein, secretory granule core protein, Hematopoietic proteoglycan core protein, Platelet proteoglycan core protein or secretory granule proteoglycan core protein. The full length amino acid sequence of human serglycin (SEQ ID NO: 13) is shown in FIG. 1. In embodiments in which the one or more markers comprise Serglycin or a fragment thereof, the Serglycin or fragment thereof may:
i) comprise an amino acid sequence of SEQ ID NO: 13 or fragment thereof;
ii) comprise an amino acid sequence NLPSDSQDLGQHGLEED (SEQ ID NO: 14);
iii) consist essentially of the amino acid sequence of SEQ ID NO: 14;
iv) consist of the amino acid sequence of SEQ ID NO: 14;

In one embodiment, the one or more markers is a metabolite selected from the group consisting of:
i) Propanoic acid (C₃H₆O₂);
ii) Hydroxybutyric acid (C₄H₆O₃);
iii) Citric acid (C₆H₈O₇);
iv) Proline (C₅H₉NO₂);
v) Valine (C₅H₁₁NO₂);
vi) 4-hydroxy-benzoic acid (C₇H₅O₃); and
vii) Anthranilic acid (C₆H₂(NH₂)(CO₂H), or a combination thereof.

As used herein, a “subject” is a mammal. The mammal can be a human, non-human primate, sheep, mouse, rat, dog, cat, horse, or any other mammals which can suffer from IBD. Typically, the subject is a human.

In one embodiment, the invention relates to a method of diagnosing impaired gastrointestinal barrier function is a subject. As used herein, “diagnosing impaired gastrointestinal barrier function is a subject” refers to determining whether a subject is suffering from impaired gastrointestinal barrier function. In one embodiment, diagnosing impaired gastrointestinal barrier function in a subject comprises determining whether a subject is suffering from leaky gut syndrome. In some embodiments, a subject suffering from leaky gut syndrome may also be suffering from active IBD. In some embodiments, a subject suffering from leaky gut syndrome may also be suffering from IBD in remission. In some embodiments, a subject suffering from leaky gut syndrome may not be suffering from IBD. A subject suffering from active IBD is a subject which is showing the symptoms of IBD. A subject suffering from IBD in remission is a subject who has suffered from the symptoms of IBD but which is not at the time of testing showing symptoms of IBD. A subject suffering from IBD in remission is suffering from quiescent IBD, and is not cured of the disease. In one embodiment, a subject suffering from IBD is a subject suffering from active IBD. In one embodiment, a subject suffering from IBD is a subject suffering from IBD in remission.

In some embodiments, a subject suffering from leaky gut syndrome may be suffering from Crohn's disease. A subject suffering from Crohn's disease may be a subject suffering active Crohn's disease, or from Crohn's disease in remission. A subject suffering from Crohn's disease in remission is a subject who has suffered from the symptoms of Crohn's disease but which is not at the time of testing showing symptoms of Crohn's disease. A subject suffering from Crohn's disease in remission is suffering from quiescent Crohn's disease, and is not cured of the disease. In one embodiment, a subject suffering from Crohn's disease is a subject suffering from active Crohn's disease. In one embodiment, a subject suffering from Crohn's disease is a subject suffering from Crohn's disease in remission.

In some embodiments, a subject suffering from leaky gut syndrome may be suffering from Ulcerative colitis. A subject suffering from Ulcerative colitis may be a subject suffering from active Ulcerative colitis, or from Ulcerative colitis in remission. A subject suffering from Ulcerative colitis in remission is a subject who has suffered from the symptoms of Ulcerative colitis but which is not at the time of testing showing symptoms of Ulcerative colitis. A subject suffering from Ulcerative colitis in remission is suffering from quiescent Ulcerative colitis, and is not cured of the disease. In one embodiment, a subject suffering from Ulcerative colitis is a subject suffering from active Ulcerative colitis. In one embodiment, a subject suffering from Ulcerative colitis is a subject suffering from Ulcerative colitis in remission.

The inventors have found that desmoglein, desmoplakin, FABP5, PON1, CD14, NCKP1, CLD1, CLD3, OCLN, SPP24, AMBP, GUC2a and SRGN, or peptides from these proteins, in tissue or body fluid of a subject, are markers of impaired gastrointestinal barrier function, such as leaky gut syndrome, in a subject.

As described herein, the inventors have found that the level of peptides of desmoglein, desmoplakin, FABP5, CD14, NCKP1, CLD1, CLD3, OCLN, SPP24, GUC2a and SRGN, are elevated, and the level of peptides PON1 and AMBP are reduced, following mass spectrometry (MS) analysis of serum samples from subjects suffering from leaky gut syndrome compared to the levels of the same peptides in serum samples of subjects not suffering from leaky gut syndrome, or suffering from leaky gut syndrome with low leak. Thus, the inventors have reasoned that by determining the level of one or more of desmoglein, desmoplakin, FABP5, PON1, CD14, NCKP1, CLD1, CLD3, OCLN, SPP24, AMBP, GUC2a and SRGN, or fragments thereof, relative to levels in, for example, healthy control subjects or subjects with leaky gut with low leak, it can be determined whether a subject is suffering from leaky gut syndrome and/or the severity of the leaky gut syndrome. As used herein, a “subject not suffering from leaky gut syndrome” is a subject who does not suffer from leaky gut syndrome. It will be appreciated that a subject not suffering from leaky gut syndrome may nonetheless be suffering from IBD. As used herein, a “healthy subject” is a subject not suffering from IBD, or impaired gastrointestinal barrier function. In another embodiment, the invention relates to a method of assessing impaired gastrointestinal barrier function in a subject. In one embodiment, the method comprising assessing leaky gut syndrome in the subject. As used herein, “assessing leaky gut syndrome in a subject” refers to determining the severity of leaky gut syndrome in a subject. Leaky gut syndrome can vary in severity, and may therefore be classified as leaky gut syndrome with low leak, or leaky gut syndrome with high leak. Leaky gut syndrome with low leak typically exhibits a confocal endomicroscopy score (CLE leakiness score) in the range of from 0 to 12.8. Leaky gut syndrome with high leak typically exhibits a confocal endomicroscopy score (CLE leakiness score) of 12.9 or above, more typically in the range of from 12.9 to 22.5. The inventors have found that the level of desmoglein, desmoplakin, CD14, NCKP1, CLD1, CLD3, OCLN, SPP24, GUC2a and/or SRGN, in serum of subjects suffering from leaky gut syndrome with high leak is elevated relative to the levels of these markers in serum of subjects suffering from leaky gut with low leak. Accordingly, the inventors reason that increased levels of desmoglein, desmoplakin, CD14, NCKP1, CLD1, CLD3, OCLN, SPP24, GUC2a and SRGN, in a subject relative to the level of the same peptide in a subject suffering from leaky gut with low leak is indicative that the subject is suffering from leaky gut syndrome with high leak.

The inventors have also found that the levels of SPP24, CD14, Occludin, claudin 3, desmoglein, desmoplakin, and FABP5 are elevated in serum of subjects suffering from severe Crohn's disease, and complicated Crohn's disease (CCD), relative to the serum of subjects suffering from Crohn's disease without complications, or Crohn's disease in remission, or relative to serum from healthy subjects. Subjects suffering from CCD experience strictures or fistulas in the gastrointestinal tract. Accordingly, the inventors reason that increased levels of SPP24, CD14, Occludin, claudin 3, desmoglein, desmoplakin, and FABP5 in a subject suffering from CD relative to the level of these peptides in a subject not suffering from CCD, such as a healthy subject, or a subject suffering from CD but not CCD, is indicative of CCD.

A subject suffering from CD but not CCD, is a subject suffering from CD in remission, or suffering from inflammatory CD (i.e. active CD).

In another embodiment, the invention relates to a method of monitoring impaired gastrointestinal barrier function in a subject. Monitoring of impaired gastrointestinal barrier function can be conducted by assessing the impaired GIT barrier function over time. In one embodiment, the method comprising monitoring leaky gut syndrome in the subject. In another embodiment, the method comprises monitoring complications of IBD in a subject, typically CCD in a subject.

Diagnosis, assessment or monitoring of impaired gastrointestinal barrier function may be made using a single marker, or using a combination of the markers described herein.

In various embodiments, the one or more markers used to diagnose, assess or monitor leaky gut syndrome are:
(a) desmoglein-1 or a fragment thereof;
(b) desmoglein-1 or a fragment thereof, desmoplakin or a fragment thereof, and FABP5 or a fragment thereof;
(c) desmoglein-1 or a fragment thereof, and desmoplakin or a fragment thereof;
(d) desmoglein-1 or a fragment thereof, and FABP5 or a fragment thereof;
(e) desmoplakin or a fragment thereof;
(f) FABP5 or a fragment thereof;
(g) PON1 or a fragment thereof;
(h) PON1 or a fragment thereof, and SPP24 or a fragment thereof;
(i) CD14 or a fragment thereof;
(j) CD14 or a fragment thereof, and SPP24 or a fragment thereof;
(k) CD14 or a fragment thereof, and NCKP1 or a fragment thereof;
(l) CD14 or a fragment thereof, and SPP24 or a fragment thereof, and NCKP1 or a fragment thereof;
(m) Guanylin or a fragment thereof;
(n) Guanylin or a fragment thereof and serglycin or a fragment thereof;
(o) Guanylin or a fragment thereof and AMBP or a fragment thereof;
(p) Guanylin or a fragment thereof, serglycin or a fragment thereof and AMBP or a fragment thereof;
(q) NCKP1 or a fragment thereof;
(r) SPP24 or a fragment thereof;
(s) SPP24 or a fragment thereof, and serglycin or a fragment thereof;
(t) Claudin-1 or a fragment thereof;
(u) Claudin-3 or a fragment thereof;
(v) Claudin-1 or a fragment thereof and claudin-3 or a fragment thereof;
(w) Occludin or a fragment therof;
(x) AMBP or a fragment thereof;
(y) Serglycin or a fragment thereof;
(z) SPP24 or a fragment thereof, CD14 or a fragment thereof, occludin or a fragment thereof, claudin-3 or a fragment thereof, FABP5 or a fragment thereof, desmoglein or a fragment thereof and desmoplakin or a fragment thereof;
(aa) Occludin or a fragment thereof, claudin-3 or a fragment thereof, and claudin-1 or a fragment thereof;
(bb) SPP24 or a fragment thereof, CD14 or a fragment thereof and guanylin or a fragment thereof;
(cc) Guanylin or a fragment thereof, claudin-3 or a fragment thereof and claudin-1 or a fragment thereof;
(dd) SPP24 or a fragment thereof and NCK1 or a fragment thereof.

In various embodiments, the one or more markers used to diagnose, assess, or monitor CCD are:
(a) desmoglein-1 or a fragment thereof;
(b) desmoglein-1 or a fragment thereof, desmoplakin or a fragment thereof, and FABP5 or a fragment thereof;
(c) desmoglein-1 or a fragment thereof, and desmoplakin or a fragment thereof;
(d) desmoglein-1 or a fragment thereof, and FABP5 or a fragment thereof;
(e) desmoplakin or a fragment thereof;
(f) desmoplakin or a fragment thereof, and FABP5 or a fragment thereof; or
(g) FABP5 or a fragment thereof.

In one embodiment, the SPP24 or a fragment thereof is a fragment of SPP24 comprising SEQ ID NO: 15 or SEQ ID No: 19

In one embodiment, the PON1 or a fragment thereof is a fragment of PON1 comprising SEQ ID NO: 20.

In one embodiment, the guanylin or a fragment thereof is a fragment of guanylin comprising SEQ ID NO: 16.

In one embodiment, the AMBP or a fragment thereof is a fragment of AMBP comprising SEQ ID NO: 27.

In one embodiment, the serglycin or a fragment thereof is a fragment of serglycin comprising SEQ ID NO: 14.

In one embodiment, the desmoglein or a fragment thereof is a fragment of desmoglein comprising SEQ ID NO: 17.

In one embodiment, the desmoplakin or a fragment thereof is a fragment of desmoplakin comprising SEQ ID NO: 18.

In one embodiment, the CD14 or a fragment thereof is a fragment of CD14 comprising SEQ ID NO: 21.

In one embodiment, the NCKP1 or a fragment thereof is a fragment of NCKP1 comprising SEQ ID NO: 22.

In one embodiment, the claudin-1 or a fragment thereof is a fragment of claudin-1 comprising SEQ ID NO: 23.

In one embodiment, the claudin-3 or a fragment thereof is a fragment of claudin-3 comprising SEQ ID NO: 24.

In one embodiment, the FABP5 or a fragment thereof is a fragment of FABP5 comprising SEQ ID NO: 25.

In one embodiment, the occludin or a fragment thereof is a fragment of occludin comprising SEQ ID NO: 26.

In one embodiment, comparing the level of the one or more markers comprises determining the level of the one or more markers.

The level of the markers described herein which are a protein or fragment thereof may be determined by any known methods for determining the level of a protein in a tissue or body fluid. The method may be a direct method, in which the level of protein or fragment thereof is determined directly, or may be determined indirectly. Examples of direct methods include immunoassay and mass spectrometry. Examples of indirect methods include determining the level of expression of mRNA for a protein or peptide.

In one embodiment, the level of the one or more markers may be determined by obtaining a sample of the tissue or body fluid from the subject. The sample may be, for example, blood, serum, plasma, faeces, tissue, urine, tears, saliva, cells, organs, bone marrow, cerebrospinal fluid, sweat, bile, pancreatic juice, etc.

In one embodiment, the sample is a body fluid. The body fluid may be blood, serum, plasma, urine, feces, saliva, gastric juice, tears, sweat, bile, pancreatic juice. Typically, the body fluid is serum.

As described herein, the inventors have found that the level of a peptide or protein comprising the amino acid sequence set forth in any of SEQ ID NOs: 1-27 in a serum sample from a subject can be used to diagnose whether a subject is suffering from impaired gastrointestinal barrier function, such as leaky gut syndrome, or to assess the severity or type of leaky gut syndrome. Thus, peptides or proteins comprising the amino acid sequence set forth in SEQ ID Nos: 1 to 27 are serum markers of impaired gastrointestinal barrier function, such as leaky gut syndrome or the severity or type of leaky gut syndrome. The ability to use a serum sample provides a relatively convenient and rapid means by which to assess or diagnose impaired gastrointestinal barrier function, such as leaky gut syndrome, in a subject. As mentioned above, prior to the present invention, no markers were available for assessing, diagnosing or monitoring impaired gastrointestinal barrier function, such as leaky gut syndrome. The markers may be used individually, or a combination of the markers may be used, to diagnose or assess impaired gastrointestinal barrier function, such as leaky gut syndrome.

In another embodiment, the sample is a tissue. The tissue may be any sample from the gastrointestinal tract, including rectum, colon, small intestinal tract. The sample may be all layers from the gastrointestinal tract, or may be the mucosal layer, or the epithelial layer.

Methods for obtaining tissue and body fluid samples from subjects are known in the art.

The sample may be processed to enhance detectability of the markers. For example, the sample may be fractionated to enrich for markers of a particular size range. In this regard, a sample may be fractionated to enrich for peptides or proteins of a particular size range. Methods for fractionation of peptides and proteins in a sample are known in the art and are described in, for example, Ly and Wasinger (2008) Proteomics 8(20): pp 4197-4208; Echan et al. (2005) Proteomics 5: pp 3292-3303; Omenn (2005) Proteomics 5: pp 3226-3245. Examples of methods for size fractionation of peptides or proteins include size exclusion chromatography, ion exchange chromatography, affinity chromatography, gel electrophoresis. The sample may be processed to enrich for nucleic acid such RNA, more typcially mRNA. Methods for enrichment of RNA, including mRNA, are known in the art and are described in Simpson R. J., ed. Proteins and Proteomics: a Lab Manual. 2003 Cold Spring Harbor Laboratory Press 926; Sambrook, J., Russet D. W., ed. Molecular Cloning: A Laboratory Manual Volume 1, 2, 3. 2001. Cold Spring Harbor Laboratory Press.

In some embodiment, a sample is enriched for markers by contacting the sample with SPP24 or a fragment thereof, or Guc2a or a fragment thereof, under conditions which permit binding of the marker to SPP24 or a fragment thereof, or Guc2a or a fragment thereof. Typically, the SPP24 or a fragment thereof, or Guc2a or a fragment thereof is immobilised on a solid support. Examples of suitable solid supports include magnetic beads, plate, or microbead.

In some embodiments, the sample is enzymatically digested. For example, the sample may be enzymatically digested with a proteolytic enzyme. An example of a proteolytic enzyme is trypsin.

In some embodiments, the sample is treated to denature proteins in the sample. For example, the sample may be treated with a protein denaturant. Examples of protein denaturants suitable for treating the sample include urea, acid, acetone, heat treatment and detergent such as sodium dodecyl sulphate (SDS).

Once a sample has been obtained from the subject, the level of the one or more markers in the sample is compared with the reference value. The term “level” refers to an indication of abundance. Thus, the “level of one or more markers” refers to an indication of the abundance of one or more markers. The level of one or more markers may be a measure of the one or more markers, such as a measure of the amount of the one or more markers per unit weight or volume. The level of one or more markers may be a ratio, such as a ratio of the amount of one or more markers in a sample relative to the amount of the one or more markers of a reference value or in a control subject.

In one embodiment, the level of the one or more markers in a sample is the concentration of the one or more markers in the tissue or body fluid. The concentration of the one or more markers may be measured in any manner that is suitable for measuring concentrations of the marker in body fluids or tissue. For example, the level of the one or more markers may be determined using mass spectrometry or immunoassay.

In one embodiment, the level of the one or more markers in a sample may be determined using mass spectrometry. Examples of suitable mass spectrometry include: ionisation sources such as EI, CI, MALDI, ESI, and analysis such as Quad, ion trap, TOF, FT or combinations thereof, spectrometry, isotope ratio mass spectrometry (IRMS), thermal ionisation mass spectrometry (TIMS), spark source mass spectrometry, Multiple Reaction Monitoring (MRM) or SRM. The mass spectrometry may be conducted in combination with 2D gel electrophoresis, high performance liquid chromatography (HPLC) or other prefractionation or enrichment techniques. Methods for quantitation of molecules by two-dimensional gel electrophoresis, HPLC and mass spectrometry such as MALDI and SELDI are know in the art and are described in, for example, Simpson R. J., ed. Proteins and Proteomics: a Lab Manual. 2003 Cold Spring Harbor Laboratory Press; Sanchez, J. C. et al. Biomedical Applications of Proteomics 2004, Wiley-Blackwell. 425. Methods such as prefractionation are known and described in Ly and Wasinger (2008) Proteomics, 8(20): pp 4197-4208. Methods such as MRM are known in the art and described in, for example, Anderson and Hunter (2006) MCP, 5(4): pp.573-589.

In one embodiment, the level of the one or more markers in a sample may be determined using MRM with a reverse-polynomial dilution (RPD) calibration or a stable-isotope dilution (SID) calibration. In one embodiment, the level of the one or more markers in a sample is determined using RPD when MRM.

The level of the one or more markers may be determined using immunoassays. An immunoassay is an assay that uses an antibody to specifically bind to an antigen (e.g. the marker). The antibody may be a polyclonal, monoclonal, Fab, F(ab)₂, scFv, diabody, scFab etc. Immunoassays using antibodies include immunoblots, western blots, Enzyme linked Immunosorbant Assay (ELISA), Enzyme immunoassay (EIA), radioimmune assay. Immunoassay methods for detection and determination of levels of an antigen are known in the art and are described in, for example, Antibodies: A Laboratory Manual (1988); Monoclonal Antibodies: Principles and Practice (2^{nd} Edition, 1986); Methods in Cell Biology: Antibodies in Cell Biology, volume 37 (Asai, ed. 1993); Basic Clinical Immunology (Stits & Terr, eds., 7^{th} ed. 1991). Generally, a sample obtained from a subject can be contacted with the antibody that specifically binds the marker. Monoclonal antibodies that specifically bind to the protein markers described herein are commercially available from, for example, Abcam, Mass., USA; Santa Cruz Biotechnology, Inc. Tex., USA). The antibody may be immobilised on a solid support such as a stick, plate, bead, microbead or array. A sample such as serum, blood, plasma, urine, or saliva is incubated with the antibodies for a period of time sufficient for the antibodies to bind the markers if present, and the mixture washed to remove unbound material. Sample bound to the antibody can then be determined by incubating the mixture with a detection agent such as, for example, a second antibody labelled with a detectable agent such as a fluorescent dye, radiolabels, enzymes (e.g. horseradish peroxidise, alkaline phosphatise, etc.), colloidal gold, etc. Alternatively, the marker in the sample can be detected using an indirect assay in which, for example, a second, labelled antibody is used to detect bound specific antibody, or in a competition or inhibition assay in which, for example, binding of the marker to a labelled specific antibody inhibits binding of the specific antibody to a detection site.

In one embodiment, an antibody which binds the marker is coupled to a detectable agent. In this embodiment, direct binding of the antibody to the marker can be detected. Suitable detectable agents are as mentioned above and include fluorescent dye, radiolabels, enzymes (e.g. horseradish peroxidise, alkaline phosphatise, etc.), colloidal gold, etc.

The level of the one or more markers in a sample may be determined indirectly by determining the expression of mRNA for the marker in a tissue sample. In this regard, there is typically a correlation between mRNA levels and protein expression. Accordingly, elevated or reduced levels of mRNA relative to a control is likely to reflect elevated or reduced levels of the protein encoded by that mRNA. The inventors therefore envisage that in some embodiments, the level of mRNA of one or more markers in tissue of a subject relative a reference value can be used to diagnose or assess IBD in a subject. Methods for assessing the levels of mRNA in a tissue sample include northern blot analysis, RT-PCR, real-time RT-PCR, array analysis. Such methods are known in the art and described in, for example, Sambrook, J., Russet D. W., ed. Molecular Cloning: A Laboratory Manual Volume 1, Chapter 7, pages 7.1 to 7.88. 2001. Cold Spring Harbor Laboratory Press; Rio, D. C. et al. RNA: A Lab Manual 2011, Cold Spring Harbor Laboratory Press.

The level of the one or more markers in the sample from the subject is compared with a reference value. The “reference value for the one or more markers” is a control value which is indicative of the level of the one or more markers in a subject, or subjects, of predetermined disease status. The predetermined disease status may be, for example, not suffering from IBD or leaky gut syndrome (e.g. healthy), suffering from active IBD but not leaky gut syndrome, suffering from IBD of predetermined severity (e.g. mild, moderate, severe, in remission), or suffering from IBD of a predetermined type, such as active Crohn's disease, Crohn's disease in remission, active Ulcerative colitis, Ulcerative colitis in remission, or not suffering from CCD.

The reference value may be a predetermined standard value. For example, the reference value may be a predetermined standard value, or a range of predetermined standard values, for a marker which represents no illness, or a predetermined type or severity of illness.

The reference value may be the level of the one or more markers in a reference sample from a subject, or a pool of subjects, not suffering from IBD or leaky gut syndrome, or suffering from IBD but not leaky gut syndrome, or suffering from leaky gut syndrome with low leak, or suffering from leaky gut syndrome with high leak, or not suffering from CCD. In some forms, the predetermined severity may be active. As used herein, “active” refers to disease which is not in remission, and may be mild, moderate or severe in severity. In some embodiments, the disease severity is as determined by a disease activity index (DAI) for CD or UC such as, for example, the Harvey-Bradshaw Index for CD, or the Ulcerative Colitis Disease Activity Index for UC.

In one embodiment, the reference value is the level of the one or more markers in the tissue or body fluid of a subject, or subjects, having a predetermined disease status.

In one embodiment, the reference value is the level of the one or more markers in a reference sample. A reference sample is a sample of tissue or body fluid for a subject, or subjects, having a predetermined disease status. A subject of predetermined disease status may be referred to as a reference subject. In one embodiment, the level of the one or more markers in a reference sample is the concentration of the one or more markers in the reference sample.

A reference sample may be from a subject not suffering from leaky gut syndrome. By comparing the level of the one or more markers in a tissue or body fluid of a subject with a level of the one or more markers from a reference sample obtained from a single subject, or a plurality of subjects, not suffering from leaky gut syndrome, it is possible to diagnose whether the subject is suffering from leaky gut syndrome. The subject not suffering from leaky gut syndrome may be suffering from IBD.

The reference sample may be from a subject suffering from leaky gut syndrome of known severity, such as leaky gut syndrome with low leak. In this regard, by comparing, for example, the level of occludin or a fragment thereof in a sample obtained from a subject suffering from leaky gut syndrome with the level of occludin or fragment thereof from a reference sample obtained from a subject, or plurality of subjects, suffering from leaky gut syndrome with low leak, it is possible to determine the whether the disease has progressed to a high leak status. In this regard, the subject has progressed to high leak status if the level of occludin or fragment thereof in sample from the subject is elevated relative to the reference sample.

In embodiments in which CCD is being diagnosed, assessed or monitored, the reference sample may be from a subject not suffering from CCD.

In various embodiments:
(a) The one or more markers is desmoglein or a fragment thereof, the reference value is the level of desmoglein or a fragment thereof in the tissue or body fluid of a subject or subjects suffering from low leak leaky gut syndrome, wherein leaky gut syndrome with high leak is diagnosed when the level of desmoglein or a fragment thereof is elevated relative to the reference value.
(b) The one or more markers is desmoglein or a fragment thereof, the reference value is the level of desmoglein or a fragment thereof in the tissue or body fluid of a subject or subjects not suffering from leaky gut syndrome, wherein leaky gut syndrome is diagnosed when the level of desmoglein or a fragment thereof is elevated relative to the reference value.
(c) The one or more markers is desmoplakin or a fragment thereof, the reference value is the level of desmoplakin or a fragment thereof in the tissue or body fluid of a subject or subjects not suffering from leaky gut syndrome, wherein leaky gut syndrome is diagnosed when the level of desmoplakin or a fragment thereof is elevated relative to the reference value.
(d) The one or more markers is FABP5 or a fragment thereof, the reference value is the level of FABP5 or a fragment thereof in the tissue or body fluid of a subject or subjects not suffering from leaky gut syndrome, wherein leaky gut syndrome is diagnosed when the level of FABP5 or a fragment thereof is elevated relative to the reference value.
(e) The one or more markers is CD14 or a fragment thereof, the reference value is the level of CD14 or a fragment thereof in the tissue or body fluid of a subject or subjects not suffering from leaky gut syndrome, wherein leaky gut syndrome is diagnosed when the level of CD14 or a fragment thereof is elevated relative to the reference value.
(f) The one or more markers is NCKP1 or a fragment thereof, the reference value is the level of NCKP1 or a fragment thereof in the tissue or body fluid of a subject or subjects suffering from low leak leaky gut syndrome, wherein leaky gut syndrome with high leak is diagnosed when the level of NCKP1 or a fragment thereof is elevated relative to the reference value.
(g) The one or more markers is NCKP1 or a fragment thereof, the reference value is the level of NCKP1 or a fragment thereof in the tissue or body fluid of a subject or subjects not suffering from leaky gut syndrome, wherein leaky gut syndrome is diagnosed when the level of NCKP1 or a fragment thereof is elevated relative to the reference value.
(h) The one or more markers is claudin-1 or a fragment thereof, the reference value is the level of claudin-1 or a fragment thereof in the tissue or body fluid of a subject or subjects suffering from low leak leaky gut syndrome, or not suffering from leaky gut syndrome, wherein leaky gut syndrome with high leak is diagnosed when the level of claudin-1 or a fragment thereof is elevated relative to the reference value.
(i) The one or more markers is claudin-1 or a fragment thereof, the reference value is the level of claudin-1 or a fragment thereof in the tissue or body fluid of a subject or subjects not suffering from leaky gut syndrome, wherein leaky gut syndrome is diagnosed when the level of claudin-1 or a fragment thereof is elevated relative to the reference value.
(j) The one or more markers is claudin-3 or a fragment thereof, the reference value is the level of claudin-3 or a fragment thereof in the tissue or body fluid of a subject or subjects suffering from low leak leaky gut syndrome, wherein leaky gut syndrome with high leak is diagnosed when the level of claudin-3 or a fragment thereof is elevated relative to the reference value.
(k) The one or more markers is claudin-3 or a fragment thereof, the reference value is the level of claudin-3 or a fragment thereof in the tissue or body fluid of a subject or subjects not suffering from leaky gut syndrome, wherein leaky gut syndrome is diagnosed when the level of claudin-3 or a fragment thereof is elevated relative to the reference value.
(l) The one or more markers is occludin or a fragment thereof, the reference value is the level of occludin or a fragment thereof in the tissue or body fluid of a subject or subjects suffering from low leak leaky gut syndrome, wherein leaky gut syndrome with high leak is diagnosed when the level of occludin or a fragment thereof is elevated relative to the reference value.
(m) The one or more markers is occludin or a fragment thereof, the reference value is the level of occludin or a fragment thereof in the tissue or body fluid of a subject or subjects not suffering from leaky gut syndrome, wherein leaky gut syndrome is diagnosed when the level of occludin or a fragment thereof is elevated relative to the reference value.
(n) The one or more markers is SPP24 or a fragment thereof, the reference value is the level of SPP24 or a fragment thereof in the tissue or body fluid of a subject or subjects suffering from low leak leaky gut syndrome, wherein leaky gut syndrome with high leak is diagnosed when the level of SPP24 or a fragment thereof is elevated relative to the reference value.
(o) The one or more markers is SPP24 or a fragment thereof, the reference value is the level of SPP24 or a fragment thereof in the tissue or body fluid of a subject or subjects not suffering from leaky gut syndrome, wherein leaky gut syndrome is diagnosed when the level of SPP24 or a fragment thereof is elevated relative to the reference value.
(p) The one or more markers is AMBP or a fragment thereof, the reference value is the level of AMBP or a fragment thereof in the tissue or body fluid of a subject or subjects suffering from low leak leaky gut syndrome, wherein leaky gut syndrome with high leak is diagnosed when the level of AMBP or a fragment thereof is reduced relative to the reference value.
(q) The one or more markers is AMBP or a fragment thereof, the reference value is the level of AMBP or a fragment thereof in the tissue or body fluid of a subject or subjects not suffering from leaky gut syndrome, wherein leaky gut syndrome is diagnosed when the level of AMBP or a fragment thereof is reduced relative to the reference value.
(r) The one or more markers is guanylin or a fragment thereof, the reference value is the level of guanylin or a fragment thereof in the tissue or body fluid of a subject or subjects suffering from low leak leaky gut syndrome, wherein leaky gut syndrome with high leak is diagnosed when the level of guanylin or a fragment thereof is elevated relative to the reference value.
(s) The one or more markers is guanylin or a fragment thereof, the reference value is the level of guanylin or a fragment thereof in the tissue or body fluid of a subject or subjects not suffering from leaky gut syndrome, wherein leaky gut syndrome is diagnosed when the level of guanylin or a fragment thereof is elevated relative to the reference value.
(t) The one or more markers is serglycin or a fragment thereof, the reference value is the level of serglycin or a fragment thereof in the tissue or body fluid of a subject or subjects not suffering from leaky gut syndrome, wherein leaky gut syndrome is diagnosed when the level of serglycin or a fragment thereof is elevated relative to the reference value.
(u) The one or more markers is serglycin or a fragment thereof, the reference value is the level of serglycin or a fragment thereof in the tissue or body fluid of a subject or subjects suffering from low leak leaky gut syndrome, wherein high leak leaky gut syndrome is diagnosed when the level of serglycin or a fragment thereof is elevated relative to the reference value.
(v) The one or more markers is propanoic acid, the reference value is the level of propanoic acid in the tissue or body fluid of a subject or subjects not suffering from leaky gut syndrome, wherein leaky gut syndrome is diagnosed when the level of propanoic acid is elevated relative to the reference value.
(w) The one or more markers is hydroxybutyric acid, the reference value is the level of hydroxybutyric acid in the tissue or body fluid of a subject or subjects not suffering from leaky gut syndrome, wherein leaky gut syndrome is diagnosed when the level of hydroxybutyric acid is elevated relative to the reference value.
(x) The one or more markers is citrate, the reference value is the level of citrate in the tissue or body fluid of a subject or subjects not suffering from leaky gut syndrome, wherein leaky gut syndrome is diagnosed when the level of citrate is elevated relative to the reference value.
(y) The one or more markers is proline, the reference value is the level of proline in the tissue or body fluid of a subject or subjects not suffering from leaky gut syndrome, wherein leaky gut syndrome is diagnosed when the level of proline is elevated relative to the reference value.
(z) The one or more markers is valine, the reference value is the level of valine in the tissue or body fluid of a subject or subjects not suffering from leaky gut syndrome, wherein leaky gut syndrome is diagnosed when the level of valine is elevated relative to the reference value.
(aa) The one or more markers is 4-hydroxy-benzoic acid, the reference value is the level of 4-hydroxy-benzoic acid in the tissue or body fluid of a subject or subjects not suffering from leaky gut syndrome, wherein leaky gut syndrome is diagnosed when the level of 4-hydroxy-benzoic acid is elevated relative to the reference value.
(bb) The one or more markers is anthranilic acid, the reference value is the level of anthranilic acid in the tissue or body fluid of a subject or subjects not suffering from leaky gut syndrome, wherein leaky gut syndrome is diagnosed when the level of anthranilic acid is elevated relative to the reference value.
(cc) The one or more markers is desmoglein or a fragment thereof, the reference value is the level of desmoglein or a fragment thereof in tissue or body fluid of a subject or subjects not suffering from CCD, wherein CCD is diagnosed when the level of desmoglein or a fragment thereof is elevated relative to the reference value.
(dd) The one or more markers is desmoplakin or a fragment thereof, the reference value is the level of desmoplakin or a fragment thereof in tissue or body fluid of a subject or subjects not suffering from CCD, wherein CCD is diagnosed when the level of desmoplakin or a fragment thereof is elevated relative to the reference value.
(ee) The one or more markers is FABP5 or a fragment thereof, the reference value is the level of FABP5 or a fragment thereof in tissue or body fluid of a subject or subjects not suffering from CCD, wherein CCD is diagnosed when the level of FABP5 or a fragment thereof is elevated relative to the reference value.
(ff) The one or more markers is desmoglein or a fragment thereof, and FABP5 or a fragment thereof, the reference value is the level of the one or more markers in tissue or body fluid of a subject or subjects not suffering from CCD, wherein CCD is diagnosed when the level of the one or more markers is elevated relative to the reference value.
(gg) The one or more markers is desmoplakin or a fragment thereof, and FABP5 or a fragment thereof, the reference value is the level of the one or more markers in tissue or body fluid of a subject or subjects not suffering from CCD, wherein CCD is diagnosed when the level of the one or more markers is elevated relative to the reference value.
(hh) The one or more markers is desmoplakin or a fragment thereof, and desmoglein or a fragment thereof, the reference value is the level of the one or more markers in tissue or body fluid of a subject or subjects not suffering from CCD, wherein CCD is diagnosed when the level of the one or more markers is elevated relative to the reference value.

The methods described herein may be used independently of other diagnostic tests, or may be used in combination with other diagnostic tests.

As used herein, the term “elevated” means more than or greater than. Typically, a level “elevated relative to the reference value” is a level that is statistically significantly more than or greater than the reference value. A level may be elevated relative to a reference value by any amount that is statistically significant more than the reference value. Typically, levels greater than 1.2 fold are significant. In various embodiments, the level may elevated by about 1.2, 1.3, 1.4, 1.5, 1.6, 1.7, 1.8, 1.9, 2.0. 3, 4, 5, 6, 7, 8, 9, or 10 fold or greater.

As used herein, the term “reduced” means less than or lower than. Typically, a level “reduced relative to the reference value” is a level that is statistically significantly less than or lower than the reference value. A level may be reduced relative to a reference value by any amount that is statistically significant less than the reference value. Typically, levels that are reduced by than 1.2 fold or more are significant. In various embodiments, the level may be reduced by at least about 0.9, 0.8, 0.7, 0.6, 0.5, 0.4, 0.3, 0.2, 0.1, 0.05, 0.01 fold.

As used herein, the term “not elevated” means not statistically significantly more than or greater than. A level that is “not elevated relative to the reference value” may be a level that is not statistically significantly more than the reference value, or is less than the reference value. Statistical significance may be determined by any methods known in the art, such as, for example, a student t test.

In one embodiment, the method of diagnosing, assessing, or monitoring comprises the further step of treating the subject for impaired gastrointestinal barrier function if the subject is diagnosed or assessed as suffering from impaired gastrointestinal barrier function. Methods for the treatment of impaired gastrointestinal barrier function which may be employed include treatment methods for IBD, CD and UC. Methods for treatment of IBD, CD and UC are known in the art and are described in, for example, D.C. Baumgart, Sandborn, W.J., Inflammatory bowel disease: clinical aspects and established and evolving therapies, The Lancet 369 (2007) 1641-1657. Treatments for UC and CD which are be suitable for treatment of impaired gastrointestinal barrier function include administration of anti-inflammatory agents such as 5-aminosalicylic acid, mesalazine, sulfasalazine, mesalamine, olsalazine, balsalazide; corticosteroids; immune modifiers such as thiopurines such as 6-mercaptopurine, azathioprine, calineurin inhibitors such as cyclosporine A, tacrolimus; methotrexate, anti-TNF agents such as infliximab, adalimumab, certolizumab, as well as dietary management.

The invention further relates to one or more diagnostic peptide selected from the group consisting of: VTVQDGNFSFSLESVK (SEQ ID NO: 16); VSAQQVQGVHAR (SEQ ID NO: 15); VNSQSLSPYLFR (SEQ ID NO: 19); NLPSDSQDLGQHGLEED (SEQ ID NO: 14); HHGPTITAK (SEQ ID NO: 27); ILLMDLNEEDPTVLELGITGSK (SEQ ID NO: 20); TGEINITSIVDR (SEQ ID. NO: 17); TGDGIQLTR (SEQ ID NO: 18); AFPALTSLDLSDNPGLGER (SEQ ID NO: 21); SENISPEEEYK (SEQ ID NO: 22); VFDSLLNLSSTLQATR (SEQ ID NO; 23); DFYNPVVPEAQK (SEQ ID NO: 24); ELGVGIALR (SEQ ID NO: 25); and NFDTGLQEYK (SEQ ID NO: 26). In one embodiment, the diagnostic peptides are synthetic peptides. Typically, the peptides are isotopically labelled. Accordingly, one embodiment provides one or more isotopically labelled peptide selected from the group consisting of: VTVQDGNFSFSLESVK (SEQ ID NO: 16); VSAQQVQGVHAR (SEQ ID NO: 15); VNSQSLSPYLFR (SEQ ID NO: 19); NLPSDSQDLGQHGLEED (SEQ ID NO: 29); HHGPTITAK (SEQ ID NO: 27); ILLMDLNEEDPTVLELGITGSK (SEQ ID NO: 20); TGEINITSIVDR (SEQ ID. NO: 17); TGDGIQLTR (SEQ ID NO: 18); AFPALTSLDLSDNPGLGER (SEQ ID NO: 21); SENISPEEEYK (SEQ ID NO: 22); VFDSLLNLSSTLQATR (SEQ ID NO; 23); DFYNPVVPEAQK (SEQ ID NO: 24); ELGVGIALR (SEQ ID NO: 25); and NFDTGLQEYK (SEQ ID NO: 26). Typically, the isotopically labelled peptide is a synthetic peptide.

The invention further relates to a panel of two or more of the markers described herein, or a panel of two of more antibodies or antigen binding fragments thereof which specifically bind the markers described herein, for use in diagnosing, assessing or monitoring a subject for impaired gastrointestinal barrier function. In one embodiment, there is provided a panel of 2 or more markers selected from the group consisting of: desmoglein or a fragment thereof; desmoplakin or a fragment thereof; SPP 24 or a fragment thereof; Paraoxonase or a fragment thereof; CD14 protein precursor or a fragment thereof; Nck-associated protein 1 or a fragment thereof; Claudin-1 or a fragment thereof; Claudin-3 or a fragment thereof; Fatty acid-binding protein 5 or a fragment thereof; Occludin or a fragment thereof; Alpha-1-microglobulin or a fragment thereof; Guanylate cyclase activator 2a or a fragment thereof; Serglycin or a fragment thereof; Propanoic acid; Hydroxybutyric acid; Citric acid; Proline; Valine; 4-hydroxy-benzoic acid; and Anthranilic acid. Typically, the panel of markers are labelled in a manner suitable for mass spectrometry. Typically, the panel of markers are isotopically labelled. Typically, the panel of markers are synthetic.

The peptides may be labelled with any isotope suitable for use in mass spectrometry. The isotope may be, for example, C⁻³ or C¹³ and N¹⁵.

In one embodiment, there is provided a panel of 2 or more antibodies or antigen binding fragment thereof, wherein each antibody or antigen binding fragment of the panel binds a marker selected from the group consisting of desmoglein or a fragment thereof; desmoplakin or a fragment thereof; SPP 24 or a fragment thereof; Paraoxonase or a fragment thereof; CD14 protein precursor or a fragment thereof; Nck-associated protein 1 or a fragment thereof; Claudin-1 or a fragment thereof; Claudin-3 or a fragment thereof; Fatty acid-binding protein 5 or a fragment thereof; Occludin or a fragment thereof; Alpha-1-microglobulin or a fragment thereof; Guanylate cyclase activator 2a or a fragment thereof; Serglycin or a fragment thereof.

The invention further relates to a composition comprising one or more peptides selected from the group consisting of: desmogelin or a fragment thereof, such as TGEINITSIVDR (SEQ ID. NO: 17); desmoplakin or a fragment thereof, such as YGDGIQLTR (SEQ ID NO: 18); paraoxonase 1 or a fragment thereof, such as ILLMDLNEEDPTVLELGITGSK (SEQ ID NO: 20); CD14 or a fragment thereof, such as AFPALTSLDLSDNPGLGER (SEQ ID NO: 21); Nck-associated protein 1 or a fragment thereof, such as SENISPEEEYK (SEQ ID NO: 22); claudin-1 or a fragment thereof, such as VFDSLLNLSSTLQATR (SEQ ID NO; 23); claudin-3 or a fragment thereof, such as DFYNPVVPEAQK (SEQ ID NO: 24); FABPS or a fragment thereof, such as ELGVGIALR (SEQ ID NO: 25); occludin or a fragment thereof, such as NFDTGLQEYK (SEQ ID NO: 26); guanylin or a fragment thereof, such as VTVQDGNFSFSLESVK (SEQ ID NO: 16); SPP 24 or a fragment thereof, such as VSAQQVQGVHAR (SEQ ID NO: 15) or VNSQSLSPYLFR (SEQ ID NO: 19); Serglycin or a fragment thereof, such as NLPSDSQDLGQHGLEED (SEQ ID NO: 14); AMBP or a fragment thereof, such as HHGPTITAK (SEQ ID NO: 27); and/or one or more compounds selected from the group consisting of Propanoic acid; Hydroxybutyric acid; Citric acid; Proline; Valine; 4-hydroxy-benzoic acid; and Anthranilic acid. In one embodiment, the peptides or compounds are isotopically labelled. The peptides may be labelled with any isotope suitable for use in mass spectrometry. The isotope may be, for example, C¹³ or C¹³ and N¹⁵.

The invention further relates to an antibody or antigen binding fragment thereof when used for diagnosing, assessing or monitoring impaired gastrointestinal barrier function, such as leaky gut syndrome or CCD, in the tissue or body fluid of a subject, wherein the antibody specifically binds a marker selected from the group consisting of (i) desmoglein or a fragment thereof; (ii) desmoplakin or a fragment thereof; (iii) SPP 24 or a fragment thereof; (iv) Paraoxonase or a fragment thereof; (v) CD14 protein precursor or a fragment thereof; (vi) Nck-associated protein 1 or a fragment thereof; (vii) Claudin-1 or a fragment thereof; (viii) Claudin-3 or a fragment thereof; (ix) Fatty acid-binding protein or a fragment thereof; (x) Occludin or a fragment thereof; (xi) Alpha-1-microglobulin or a fragment thereof; (xii) Guanylate cyclase activator 2a or a fragment thereof; (xiii) Serglycin or a fragment thereof. Monoclonal antibodies that specifically bind to the protein markers described herein are commercially available from, for example, Abcam, Mass., USA; Santa Cruz Biotechnology, Inc. Tex., USA).

The invention further relates to kits for diagnosing, assessing or monitoring impaired gastrointestinal barrier function in the tissue or body fluid of a subject. The kits can be used to carry out the method of the invention. In one form, the kit comprises antibodies or antigen binding fragments thereof which specifically bind to one or more markers described herein. In another form, the kit comprises nucleic acid such as primers for amplifying one or more markers described herein using, for example, RT-PCR, or probes, for detection of mRNA. In another form, the kits comprises one or more of the protein or peptide markers described herein.

The kits may comprise a control sample such as a sample from a subject or subjects not suffering from impaired gastrointestinal barrier function, such as leaky gut syndrome, or not suffering from CCD, or suffering from impaired gastrointestinal barrier function of a predetermined severity, such as leaky gut syndrome with high or low leak, or suffering from CD. Kits may comprise a solid support on which the marker, or antibody which specifically binds to the marker, is immobilised. Kit which comprise an antibody to the marker may comprise a second antibody conjugated to a detectable group. Kits comprising nucleic acids may comprise fluorescent labels for detection of hybridisation.

In some embodiments, the marker, or antibody or antigen-binding fragments thereof which specified bind the marker, nucleic acids or peptides for detecting the marker, may be immobilised on a solid support. A “solid support” is any non-aqueous matrix, which is chemically inert and insoluble in an assay solution, to which a molecule, such as an antibody or peptide, can adhere or be conjugated. Any suitable solid support can be used, such as beads, microparticles, glass, polymers such as polysaccharides (e.g., agarose), polyacrylamides, polystyrene, polyvinyl alcohol, silicones, magnetic or chromatographic matrix particles, the surface of an assay plate (e.g., microtiter wells), pieces of a solid substrate material or membrane (e.g., plastic, nylon, paper), etc. In some embodiments, the solid support can be the interior of an assay container, such as the well of an assay plate; a dipstick; a particle inside an assay container, etc. The attachment or linkage of the antibody or marker to the solid support can be by any suitable means, such as by electrostatic attraction, affinity interaction, hydrophobic interaction, covalent bonding, etc.

As described in the Examples, the inventors have found that peptides from SPP24 and GUC2a immobilised on magnetic beads can be used as a bait to isolate from tissue or body fluid markers that are indicative of impaired gastrointestinal barrier function.

Accordingly, in one aspect, there is provided a kit for diagnosing or assessing or monitoring impaired gastrointestinal barrier function in the tissue or body fluid of a subject, the kit comprising SPP24 or a fragment thereof, and/or GUC2a or a fragment thereof, immobilised on a solid support.

In another aspect, there if provided a kit for diagnosing or assessing or monitoring impaired gastrointestinal barrier function in the tissue or body fluid of a subject, comprising one or more markers immobilised on a solid support, wherein the one or more markers are selected from the group consisting of (i) desmoglein or a fragment thereof; (ii) desmoplakin or a fragment thereof; (iii) SPP 24 or a fragment thereof; (iv) Paraoxonase or a fragment thereof; (v) CD14 protein precursor or a fragment thereof; (vi) Nck-associated protein 1 or a fragment thereof; (vii) Claudin-1 or a fragment thereof; (viii) Claudin-3 or a fragment thereof; (ix) Fatty acid-binding protein or a fragment thereof; (x) Occludin or a fragment thereof; (xi) Alpha-1-microglobulin or a fragment thereof; (xii) Guanylate cyclase activator 2a or a fragment thereof; (xiii) Serglycin or a fragment thereof; (xiv) Propanoic acid; (xv) Hydroxybutyric acid; (xvi) Citric acid; (xvii) Proline; (xviii) Valine; (xix) 4-hydroxy-benzoic acid; and (xx) Anthranilic acid.

In another aspect, there if provided a kit for diagnosing or assessing or monitoring impaired gastrointestinal barrier function in the tissue or body fluid of a subject, comprising one or more antibodies or antigen binding fragments thereof, wherein the one or more antibodies or fragments thereof are immobilised on a solid support, and wherein each antibody or antigen binding fragment thereof specifically binds to a marker selected from the group consisting of (i) desmoglein or a fragment thereof; (ii) desmoplakin or a fragment thereof; (iii) SPP 24 or a fragment thereof; (iv) Paraoxonase or a fragment thereof; (v) CD14 protein precursor or a fragment thereof; (vi) Nck-associated protein 1 or a fragment thereof; (vii) Claudin-1 or a fragment thereof; (viii) Claudin-3 or a fragment thereof; (ix) Fatty acid-binding protein or a fragment thereof; (x) Occludin or a fragment thereof; (xi) Alpha-1-microglobulin or a fragment thereof; (xii) Guanylate cyclase activator 2a or a fragment thereof; and (xiii) Serglycin or a fragment thereof.

In one aspect, there is provided a kit when used for diagnosing or assessing or monitoring impaired gastrointestinal barrier function in the tissue or body fluid of a subject, comprising: (a) one or more markers immobilised on a solid support, wherein the one or more markers is selected from the group consisting of (i) desmoglein or a fragment thereof; (ii) desmoplakin or a fragment thereof; (iii) SPP 24 or a fragment thereof; (iv) Paraoxonase or a fragment thereof; (v) CD14 protein precursor or a fragment thereof; (vi) Nck-associated protein 1 or a fragment thereof; (vii) Claudin-1 or a fragment thereof; (viii) Claudin-3 or a fragment thereof; (ix) Fatty acid-binding protein or a fragment thereof; (x) Occludin or a fragment thereof; (xi) Alpha-1-microglobulin or a fragment thereof; (xii) Guanylate cyclase activator 2a or a fragment thereof; (xiii) Serglycin or a fragment thereof; (xiv) Propanoic acid; (xv) Hydroxybutyric acid; (xvi) Citric acid; (xvii) Proline; (xviii) Valine; (xix) 4-hydroxy-benzoic acid; and (xx) Anthranilic acid; or (b) one or more antibodies or antigen binding fragments thereof immobilised on a solid support, wherein each antibody or antigen binding fragment thereof specifically binds a marker selected from the group consisting of (i) desmoglein or a fragment thereof; (ii) desmoplakin or a fragment thereof; (iii) SPP 24 or a fragment thereof; (iv) Paraoxonase or a fragment thereof; (v) CD14 protein precursor or a fragment thereof; (vi) Nck-associated protein 1 or a fragment thereof; (vii) Claudin-1 or a fragment thereof; (viii) Claudin-3 or a fragment thereof; (ix) Fatty acid-binding protein or a fragment thereof; (x) Occludin or a fragment thereof; (xi) Alpha-1-microglobulin or a fragment thereof; (xii) Guanylate cyclase activator 2a or a fragment thereof; (xiii) Serglycin or a fragment thereof.

Typically, the kits comprise instructions for use.

A further aspect provide a method for determining whether a compound in the tissue or body fluid of a subject is an indicator that the subject is suffering from impaired gastrointestinal barrier function, the method comprising:
(a) contacting tissue or body fluid of a subject suffering from impaired gastrointestinal barrier function with SPP24 or a fragment thereof or guanylin or a fragment thereof;
(b) comparing the compounds which bind the SPP24 or a fragment thereof, or guanylin or a fragment thereof, with the compounds from tissue or body fluid of a subject suffering from low or no impaired gastrointestinal barrier function which bind SPP24 or a fragment thereof or guanylin or a fragment thereof;
(c) identifying compounds which bind to the SPP24 or fragment thereof or GUC2a or fragment thereof and which differentiate between the subjects having impaired gastrointestinal barrier function and the subjects having low or no impaired gastrointestinal barrier function.

In the claims which follow and in the preceding description of the invention, except where the context requires otherwise due to express language or necessary implication, the word “comprise” or variations such as “comprises” or “comprising” is used in an inclusive sense, ie. to specify the presence of the stated features but not to preclude the presence or addition of further features in various embodiments of the invention.

It must be noted that as used herein and in the appended claims, the singular forms “a”, “an”, and “the” include plural reference unless the context clearly dictates otherwise. Thus, for example, a reference to “a peptide” includes a plurality of such peptides, and so forth. Unless defined otherwise, all technical and scientific terms used herein have the same meanings as commonly understood by one of ordinary skill in the art to which this invention belongs.

The invention will now be further described by way of reference only to the following non-limiting examples. It should be understood, however, that the examples following are illustrative only, and should not be taken in any way as a restriction on the generality of the invention described above.

### EXAMPLES

### Example 1

### Identification of Diagnostic Markers for Differentiation Between Healthy, Low Leak and High Leak Leaky Gut

Compromised epithelial integrity in the gastrointestinal tract is linked to dysfunction of the mucosal barrier. By cause or effect, this provides opportunistic organisms and their products capacity to cross-over into the blood stream. ‘Leak’ can trigger an inflammation response, which over time can become chronic resulting in gastrointestinal complications such as IBD. There is no single test that can reliably diagnose intestinal mucosal barrier function, especially in active IBD; and no biomarkers that measure impaired epithelial cell integrity.

SPP24 and GUC2a are diagnostic markers of IBD. The bioactive peptides were used as bait to explore protein changes in high and low leak patients suffering from IBD. Modulated proteins in patients with ongoing intestinal damage may be able to predict for the development of complications (inflammation, structuring, and fistulas) and predict for the need to escalate treatment.

### Methods

1. Serum proteomic profiling of 25 colonoscopy patients with corresponding confocal endomicroscopy score (CLE leakiness score), C-Reactive Protein, Erythrocyte sedimentation rate, and diagnosis (UC, CD, Healthy) was performed using label-free Liquid Chromatography-Tandem Mass Spectrometry (LC-MS/MS) to identify significant changes in proteins correlating to leakiness. Two ul of each of the patients serum was brought up into 50 ul of Ammonium bicarbonate pH8 and digested overnight with trypsin at a ratio of protein : enzyme of 100 :1. C18 membrane “stage-tips” from Thermoscientific where used to enrich and clean the serum prior to MS analysis. The data was then analysed using ProgenesisQl for proteomics as described in Wasinger et.al., 2015 (Low Mass Blood Peptides Discriminative of Inflammatory Bowel Disease (IBD) Severity: A Quantitative Proteomic Perspective.Wasinger V C, Yau Y, Duo X, Zeng M, Campbell B, Shin S, Luber R, Redmond D, Leong R W. Mol Cell Proteomics. 2016 Jan; 15(1):256-65.)

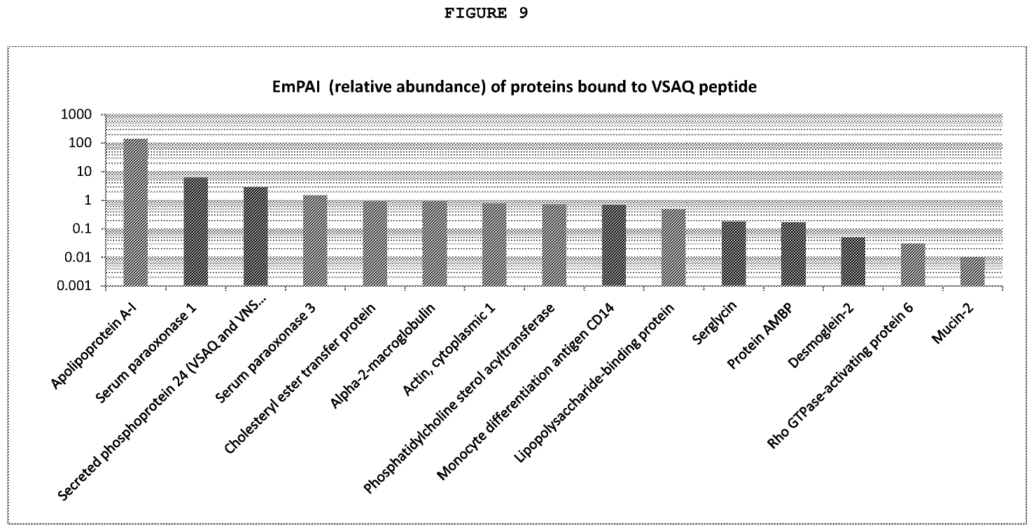

2. SPP24 bioactive peptide (VSAQQVQGVHAR) or SPP24 antibody (recognising unspecified major portion of the protein), as well as the GUC2a bioactive peptide (VTVQDGNFSFSLESVK) were used as ‘bait’ attached to magnetic bead to acquire binding partners representative of tissue integrity (SPP24-known to be associated with the endoplasmic reticulum and ER stress response) and mucosal function (GUC2a associated with mucosal barrier and goblet cells). These were identified by LCMS/MS. Tosylactivated magnetic beads (Thermo Scientific, Ill., USA) were used as per manufacturer's instructions. 100 μg synthetic SPP24 peptide, or SPP24 Antibody, was bound to the magnetic beads under basic pH conditions. Beads were washed in a basic solution containing Albumin to block binding sites. After washing to remove albumin, 50 ul patient serum (equivalent to 2.8 mg of protein) was then added to the beads and incubated overnight such that binding partners would occupy competitively the binding sites of the SPP24 peptide. Binding partners were then released following further washing to remove unbound proteins. These samples were then digested overnight as previously described and analysed s using an Orbi-trap MS instrument The relative amount of binding of various proteins to SPP24 peptide VSAQQVQGVHAR (SEQ ID NO: 15) was determined by incubating peptide coated magnetic beads with 50 ul (2.8 mg) of serum. The relative amounts of binding were determined for the serum proteins: apolipoprotein A-1, serum paraoxonase 1, SPP24, paraoxonase 3, cholesteryl ester transfer protein, alpha-2-macroglobulin, actin, phosphatidylcholine sterol acyltransferase, monocyte differentiation antigen CD14, LPS-binding protein, serglycin, AMBP, desmoglein-2, Rho GTPase-activating protein 6 and mucin-2. The emPAI for each protein was calculated according to Ishihama et al. (2005) Molecular & Cellular Proteomics 4, 1265-1272. The results are shown in FIG. 9.

3. Relative multiple reaction monitoring was used to quantitate the amounts of novel markers present in a mix of individual patients with known CLE scores, and pools of patients (high leak, low leak and control) in a proof of principle and method development project, as described in Reverse-polynomial dilution calibration methodology extends lower limit of quantification and reduces relative residual error in targeted peptide measurements in blood plasma.Yau Y Y, Duo X, Leong R W, Wasinger V C. Mol Cell Proteomics. 2015 Feb; 14(2):441-54.

### Results

### 1. Shotgun Proteomics

Physiological leak is ˜7. Proteomic profiling in whole serum identified 109 proteins in low leak IBD patients (CLE 0-9.6) and 25 proteins in high leak (CLE 12.9-22.5) modulated in abundance. Enrichment studies revealed an association to wound healing, lipid binding, regulation of response to external stimuli and adhesion.

### 2. Bait Study

Binding partners of SPP24 for peptide and antibody baited proteins correlated quite closely. Enrichment studies looking at associated diseases revealed the binding partners for both the SPP24 and GUC2a study had high probability for association to gastrointestinal disease and organismal injury, while there was also an enriched association for cell-to-cell signalling, cell interaction and movement and lipid metabolism; and the top networks indicated an association of these proteins to gastrointestinal disease, cancer, connective tissue, and cell death.

Guc2a binding partners were closely associated with cytoskeletal/intermediate filaments and lipid transport; while SPP24 binders associated with ion binding and response to wounding.

### 3. MRM Analysis

Significant changes in protein abundance were assessed by MRM for 13 proteins shown in Table 1. Bait proteins are highlighted.

TABLE 1

| Abbrev | Protein Name | Trend |
|---|---|---|
| DSG1 | desmoglein | ↑ high leak patients |
| DESP | desmoplakin | ↑ leak patients |
| FABP5 | fatty acid-binding protein 5 | ↑ leak patients/↑ compl. CD |
| PON1 | paraoxonase | ILL . . . peptide ↓ in high leak |
| | | patients/↓ high leak CD |
| CD14 | CD14 protein precursor | ↑ high leak patients |
| NCKP1 | Nck-associated protein 1 | ↑ high leak patients |
| CLD1 | claudin-1 | ↑ high leak patients |
| CLD3 | claudin-3 | ↑ high leak patients |
| OCLN | occludin | ↑ high leak patients |
| SPP24 | secreted phosphoprotein 24 | ↑ high leak patients |
| AMBP | alpha-1-microglobulin | ↓ in high leak patients |
| GUC2a | guanylate cyclase activator 2a | ↑ high leak patients |
| SRGN | serglycin | ↑ high leak patients |

574 proteins were identified using the shotgun proteomics, with a final group of 80 proteins identified as significantly changing between low CLE (0-9.6) and high CLE score (12.9-22.5).

MRM requires unique signature transitions representing a peptide and therefore the protein. Peak area of each peptide was divided by control patient's area to estimate fold change in abundance of that peptide. Where described, individual patients high and low leak were also compared.

### 3.1 Proteins Involved in Cholesterol and Fatty Acid Regulation (LXR Pathway)

There is a difference between low and high leak that is peptide dependent.

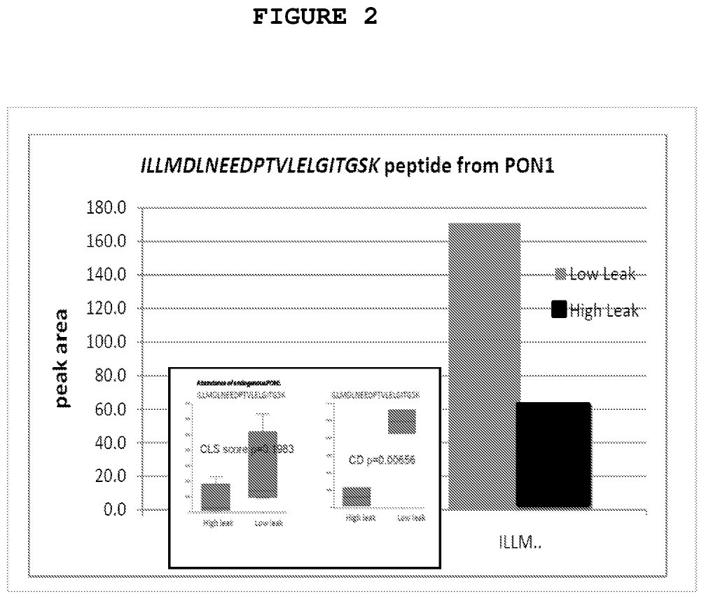

Pooled patient samples using methanol precipitation to remove low mass proteins and salts in serum prior to measuring peptides shows decrease in ILLM peptide from PON1 in high leak pool compared to low leak pool (FIG. 2). The ILLM peptide has been measured in unfractionated individual serums and shows decrease in high leak compared to low leak patients particularly for CD patients (inset of FIG. 2). There is variability in the peptides from this protein with a second peptide VVAE . . . peptide increasing in high leak while ILLM peptide decreases compared to low leak. This may be related to 3D exposure/presentation of the peptides to our digesting enzyme used for this analysis.

3.2 Proteins Associated with T3SS, Phagosome and Autophagy

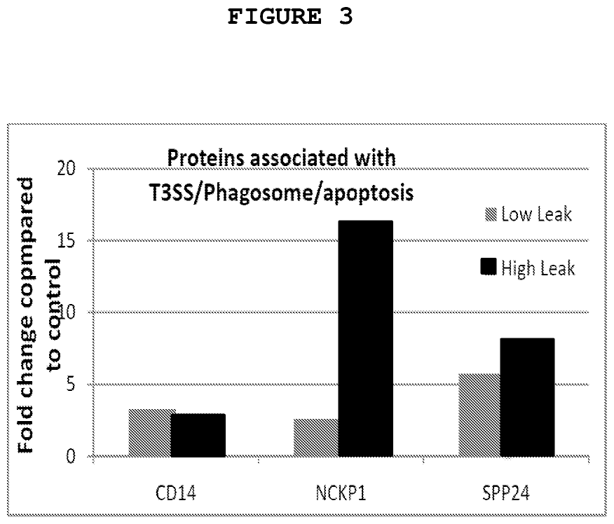

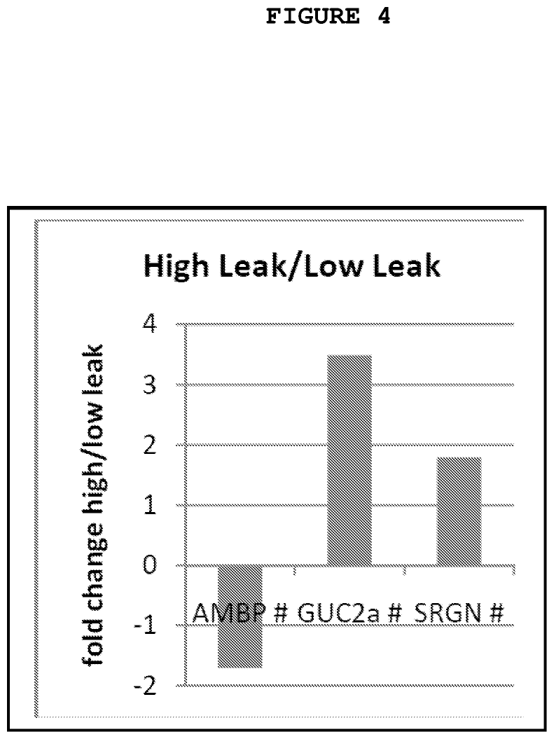

In summary, there is an increasing amount of each of CD14, NCKP1 and SPP24 proteins correlating to increased leak (FIG. 3). SPP24 binding partners show higher levels of AMBP protein in low leak compared to high leak (FIG. 4); while GUC2a and Serglycin show higher levels present in higher leak compared to lower leak patients (FIG. 4).

3.3 Proteins Associated with Paracellular Integrity and Actin Nucleation

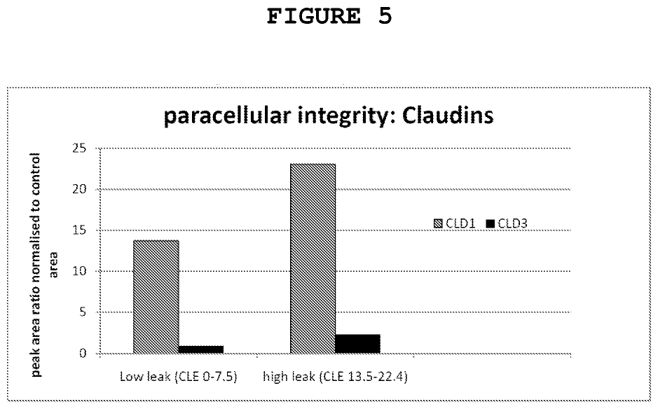

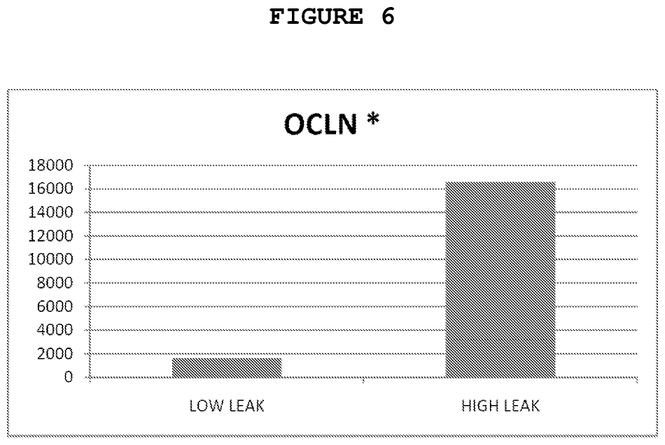

There is an increase in Claudin 1 and 3 with increased leakage compared to control patients (FIG. 5). Occludin is also increased in high leak patients compared to low leak Patients (FIG. 6). There is also 2 fold increase of CLD3 in CD severe compared to remission and 19 fold increase in Occludin in UC severe compared to remission (Data not shown).

There is an increase in desmoglein 1 in subjects suffering from high leak leaky gut syndrome compared to subjects suffering from low leak leaky gut syndrome (FIG. 7).

### 3.4 Protein Involved in Complications of Crohns Disease.

### Background:

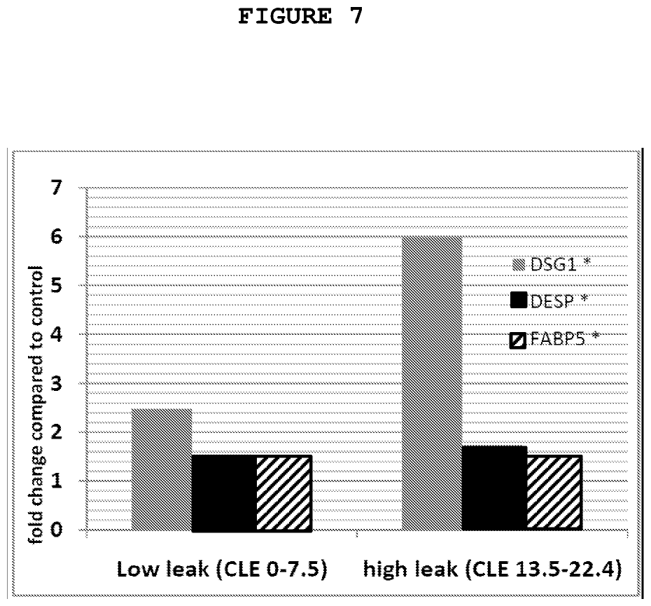

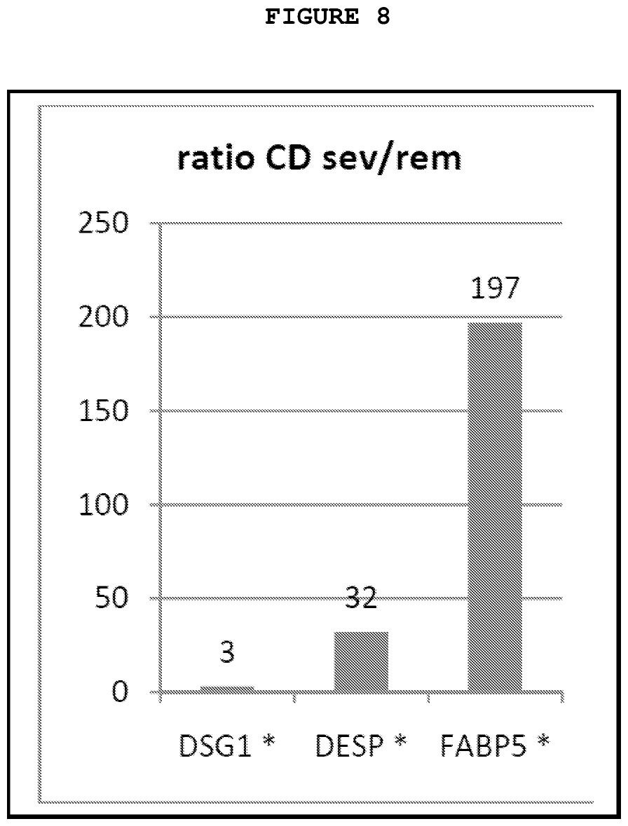

Half of all Crohn's disease (CD) patients will experience a stricturing or fistulising complication (CCD) and no current biomarker can predict this natural history. We used discovery and targeted proteomics to interrogate the differences in the low-mass (<25kDa) blood serum fraction between CD behavioural phenotypes. 172 serum proteins were modulated in CCD versus ICD by LC-MS/MS (P<0.05, q<0.01), annotating to pathways of epithelial barrier homeostasis (P<0.01). A 3-protein assay developed from discovery proteomics data consisting of Desmoglein-1, Desmoplakin and Fatty Acid-Binding Protein 5 (FABP5) distinguished CCD from all other groups (P=0.041), correlated with ESR (r=0.492, P=0.032) and discriminated complication in CD (70% sensitivity and 72.5% specificity at 1.907, AUC=0.777, P=0.007) (FIG. 8). An MRM assay secondarily confirmed increased FABP5 levels in CCD (P<0.001) Desmoglein and Desmoplakin are desmosomal proteins essential to the mechanical linkage of intestinal epithelial cells.FABPS regulates the expression of a number of cytokines and antibacterial peptides by delivering fatty acid ligands to target nuclear receptors, and interacts with Toll-Like Receptor (TLR) activation to form part of the innate immune response (FIG. 7C). FABP family members FABP1 and 2 have also been shown to predict poorer clinical outcomes in necrotizing enterocolitis. Taken together, DSG1, DSK and FABP5 may therefore be involved in the progressive intestinal injury associated with the development of CD complications.

In summary, these data show there is an increase in Desmoglein-1, Desmoplakin and Fatty Acid-Binding Protein 5 in leaky patients compared to healthy control patients. Furthermore the levels of the peptides are increased in CD patients diagnosed with severe disease and from previous work; patients with complications (fistulas).

### Conclusion:

Proteins are significantly modulated between low leak compared to high leak serum profile; thus making differentiation a possibility. The likelihood of these markers correlating to management of leak and mucosal and barrier restitution in the management of IBD and other conditions where leak contributes to disease (heart disease, dementia, alzheimers for eg.) is a reasonable prospect. We have measured differences in the peptides from the tissue integrity biomarker panel from individual patients and pooled patients to make these assessments. These markers should aid in the determination of mucosal healing and clinical remission; an emerging treatment endpoint for IBD sufferers.

### 4. Further Confirmation of Markers in Complicated Crohn's Disease

### Experimental Procedures

### Study Population & Sample Collection

Serum samples were obtained from subjects recruited from Concord Repatriation General Hospital and Bankstown-Lidcombe Hospital, Sydney Australia. CD, UC and RA subjects were recruited from IBD and rheumatology ambulatory clinics respectively, and controls from patients without gastrointestinal diseases who underwent endoscopy (eg. for screening or exclusion of gastrointestinal bleeding) with normal findings. To test biomarker specificity, RA patients were selected as positive inflammatory controls as the disease shares certain Th1/17 response pathways with CD . IBD diagnoses were confirmed by histological and endoscopic criteria and RA by rheumatoid arthritis classification criteria of at least six months duration. All CD subjects had their behavioral phenotype confirmed by a gastroenterologist with radiologic and/or endoscopic evidence within 30 days from blood sampling as part of their routine care. CCD was defined as presence of active intestinal complications (untreated/balloon dilated strictures and non-healed abscess/fistulas). ICD subjects with concomitant perianal disease were excluded as a potential confounder. Disease-specific activity indices for CD, UC, and RA (Crohn's Disease Activity Index (CDAI), Partial Mayo, and 28-Joint Disease Activity Score (DAS28), respectively) with paired biochemical markers of inflammation (CRP and Erythrocyte Sedimentation Rate (ESR)) were collected. 20 mL of peripheral blood were drawn from consenting subjects in two 20 mL SST tubes. Serum was separated from blood cells by centrifugation at 1400 rpm immediately after collection and frozen at −80° C. All participants gave written informed consent.

### Extraction of Low-Mass Serum Fraction

In-solution electrophoresis was used to partition the 1-25kDa protein component from serum samples as previously described.(Ly L, Wasinger V C. Proteomics. 2008 Oct; 8(20):4197-208 17; Wasinger et al. Molecular & cellular proteomics : MCP. 2016 Jan; 15(1):256-65.) Briefly, Pooled serum samples were created by combining 30 μL aliquots of individual patient samples based on CD behavioral phenotype, and protease inhibitor (Roche, Basel, Switzerland) was added to pooled serum samples according to manufacturer recommendations and treated serums were then diluted with 150 μL of 180 mM Tris/20 mM EACA/4M urea buffer, pH 10.2; in a 1:1 ratio. Protease inhibited serum samples were loaded onto a ProteomeSep electrophoresis instrument (NuSep, Sydney, Australia). Serum samples were separated into 1-25 kDa, 26-45 kDa, 46-65 kDa, and 66-125 kDa partitions and the 1-25 kDa (low-mass) fraction was collected for LC-MS/MS.

### Label-Free LC-MS/MS

Low-mass fraction serum samples were prepared with C18 stage tips (Thermo Scientific, IL, USA) according to manufacturer recommendations with the exception of an 80% Acetonitrile, 0.1% formic acid elution buffer. Dried peptides were re-solubilized in 50 μL of 50 mM NH4HCO3, pH 8.0. Trypsin was then added to the samples at an enzyme to protein ratio of approximately 1:100 and incubated overnight at 37° C. Following digestion, 4 μL of formic acid was added to stop the reaction and samples were dried. Samples were then resuspended in 10 μL 2% CH₃COOH, 0.1% formic acid prior to LC-MS/MS. One microliter (10%) of each sample was injected onto the nano-LC for analysis. An LTQ-FT Ultra mass spectrometer (Thermo Electron, Bremen, Germany) was used to analyze the serum samples. The sample contents were separated by nano-LC using an Ultimate 3000 HPLC and autosampler (Dionex, Amsterdam, Netherlands). Samples were loaded onto a micro C18 pre-column (500 μm×2 mm, Bruker, Calif., USA) with Buffer B (98% H2O, 2% CH₃CN, 0.1% TFA) at 10 μL min-1. After a 4-min wash the pre-column was switched (Valco 10 port valve, Dionex) into line with a fritlessnano column (75 μm i.d×10 cm) containing reverse-phase C18 media (5 μm, 200 Å Magic, Bruker). Peptides were eluted using a linear gradient of Buffer A (98% H2O, 2% CH₃COOH, 0.01% HFBA) to Buffer B (98% CH₃CN, 2% H2O, 0.01% HFBA) at 250 nL min-1 over 60 min. The column tip was positioned ˜0.5cm from the heated capillary (T=200° C.) of the LTQ-FT and 1800V was applied to a low volume tee (Upchurch Scientific, Wash., USA). The instrument was operated in Data-Dependent Acquisition (DDA) mode with positive ions generated by electrospray. A survey scan over a 350-1750 mass-to-charge ratio (m/z) range was acquired in the FT ICR cell. Collision-induced dissociation was used by the linear ion trap in which up to 8 of the most abundant ions (>2000 counts) with charge states of ≥(M+2H)+2 were successively isolated and fragmented. Mass-tocharge ratios selected for MS/MS were dynamically excluded for 60 seconds. All samples were analysed in technical triplicates.

### LC-MS/MS Spectra Analysis

LTQ-FT *.RAW data files were imported into Progenesis LC-MS v4.0 (Progenesis QI) (Waters, Mass., USA). Mass spectra from each run were aligned to a reference sample by matching consistent m/z ions by retention time in a pair-wise fashion. Quantitation of ions was achieved by gain factor calculation (logscale abundance ratio scaling factor) against the reference sample. Within-group CV % of ions was first evaluated and ions that were significantly changing within the group by one-way Analysis of Variance (ANOVA) (P<0.05) were discarded from subsequent between-group analyses. Statistical comparisons between groups were performed by one-way ANOVA with multiple testing correction by FDR q value significance set at <0.01(18). Mass spectra of significantly modulating ions were then target-decoy searched against the Swiss-prot protein knowledgebase (Release 2011_04, Apr 2011, containing >526,900 sequence entries) using the Mascot v2.3 search engine (Matrix Science, Mass., USA) with 5 ppm (peptide) and 0.5 Da (ms/ms) error tolerance settings. Mascot search parameters were set to “Allspecies”, with no enzyme and variable modifications to: cysteine (acrylamide); methionine (oxidation); serine, threonine, and tyrosine (phosphorylation). Peptides with anion scores >20 were considered for identification. Peptide-sequence matches and protein inferences were then mapped back to quantitative ion data and summary protein-level statistics were calculated (fold change, one-way ANOVA P value, q value and power).

### Functional Annotation Analysis

Functional annotation analysis was performed with DAVID v6.7 (The Database for Annotation, Visualization, and Integrated Discovery, NIH). A homo sapien gene list was used as the background comparator for the submitted dataset and the functional annotation chart and functional annotation clustering tools were examined to determine enriched biological features in the LC-MS/MS data. For the functional annotation chart an EASE score (modified Fisher's exact test) cutoff of <0.01 after Benjamini- Hochberg (BH) correction was set as the significance threshold for enrichment of each identified Gene Ontology (GO) term (Huang da W et al. Nature protocols. 2009; 4(1):44-57). For functional annotation clustering, the identified biological module groups were evaluated by their enrichment score (determined by -log transformation of the mean of all individual P-values for terms within the cluster), and the significance of the module's enrichment was determined by a BH-corrected EASE score of <0.01 for their top GO term (by EASE score) within the group (Huang da W et al. Nature protocols. 2009; 4(1):44-57). Enrichment values of individual GO terms are reported as fold change, EASE scores and Benjamini-Hochberg corrected P-values, and biological module clusters as enrichment scores (Huang da W et al. Nature protocols. 2009; 4(1):44-57). Three proteins were then selected as preliminary candidates for qualification phase immunoblot and MRM assay development.

### MRM

A NCI-CPTAC Tier 2 MRM assay was developed for FABPS as an essential part of the NCI-FDA biomarker pipeline workflow (Carr SA et al. Molecular & Cellular Proteomics. 2014 March 1, 2014; 13(3):907-17). Briefly, LC-MS/MS sequenced FABPS peptides were searched against the National Center for Biotechnology Information (NCBI) Protein BLAST database for uniqueness, and a sequence-specific precursor/fragment ion profile (transition list) was developed for a proteotypic peptide in Skyline SRM environment v1.4 (MacCoss Lab, UW). The MRM method was then validated by extensive iterative experimentation. Tier-2 level quantification was performed using a Reverse- Polynomial Dilution (RPD) calibration (Yau YY, et al. Molecular & Cellular Proteomics. 2014 December 9, 2014). Serum samples were analyzed on a 4000QTrap (ABSCIEX, Mass., USA) mass spectrometer coupled to a Dionex Ultimate 3000 liquid chromatograph (Thermo Fisher Scientific, Mass., USA). Pertinent assay information has been reported in compliance with NCI-CPTAC and the National Heart, Lung and Blood Institute's (NHLBI) Proteomics Centers' recommendations (Supplementary Experimental Procedures) (Carr SA et al. Molecular & Cellular Proteomics. 2014 March 1, 2014; 13(3):907-17).

### Immunoblot Assay

Dot blots for Desmoglein-1 (DSG1),Desmoplakin (DSK), and Fatty Acid-Binding Protein 5 (FABPS) proteins were performed in serum samples following standard procedures (Abcam®. Dot Blot Protocol May 5th 2014. Available from: http://www.abcam.com/ps/pdf/protocols/Dot%20blot%20protocol.pdf). Briefly, 5 μl of individual patient serums were dotted onto PVDF membranes and dried overnight. Membranes were blocked in milk blotto (10% milk, 10 mM tris-HCl pH 7.5, 0.9% NaCl) for 24 h before incubation in primary antibody (1 mg/mL) for 3 h (DSG1:1:500), DSK 1:1000, FABPS 1:200) and secondary antibody (anti-rabbit IgG) for 1 h (1 mg/mL diluted 1:1000 in blott). Membranes were washed in chemiluminescence buffer, incubated in chemiluminescent substrate to enhance the signal and then developed. All antibodies were purchased from Abcam (Cambridge, UK). Quantitation was performed using ImageJ 1.48v (NIH).

### Experimental Design and Statistical Rationale

This study was conducted in accordance with the biomarker development pipeline outlined by Rifai et al (Rifai N, et al. Nat Biotech. 2006 08//print; 24(8):971-83). The 2013 NCI-FDA workshop statistical model used to determine verification phase sample size (based on a biomarker candidate signal difference of greater than 5.0 standard deviations between groups and >80% power in verification phase) (Skates SJ, et al. Journal of proteome research. 2013 Dec 6; 12(12):5383-94). Values are expressed as mean±SEM. All variables were evaluated for normality of distribution using the Shapiro-Wilk test of normality and the appropriate statistical test consequently applied: One-way ANOVA was used to compare biomarker values between categorical independent variables with gaussian distributions and Kruskal Wallis H test for those with non-gaussian distributions. A Pearson's product-moment correlation and Spearman's rank-order correlation were used for equivalent continuous variables. Independent variables that met an association probability of P<0.1 were then entered into a multiple regression analysis using the enter method to determine their predictive ability for biomarker values with all other clinical factors held constant. Discriminant function analysis was used to determine discriminability of DSG1, DSK and FABPS for CCD and the subsequent discriminant function coefficients were then used to weight each protein value contribution to a combined biomarker panel score—hereafter referred to as the Serum Epithelial Components (SEC) score. Receiver Operating Characteristic (ROC) was used to determine classification ability of biomarker values and SEC score for CCD. After applying a Shapiro-Wilk test of normality on the difference between paired samples, a paired t-test was used to evaluate intra-individual variations in biomarker concentrations in the longitudinal cohort. SPSS 20 (IBM) was used for statistical analyses. GraphPad Prism 6 (GraphPad Software, Calif., USA) and TIBCO Spotfire (Tibco, Mass., USA) were used for graphical presentation of results.

Further details on experimental procedures, statistical analyses and SEC score calculation are provided in Supplementary Materials. The LC-MS/MS data in this manuscript has been deposited at the PRIDE proteomics data repository (The European Bioinformatics Institute, Cambridge, UK) with the dataset identifier PXD001821 at (http://www.ebi.ac.uk/pride/archive/). The Multiple Reaction Monitoring (MRM) Proteomics dataset has been deposited at PeptideAtlas (The Institute for Systems Biology, Wash., USA) with the identifier PASS00661 (http://www.peptideatlas.org/PASS/PASS00661).

### Results

One hundred and thirteen subjects consented to this study. Samples from 12 control subjects were excluded due to significant findings at endoscopy (ie. ulcers, atypia), and 7 CD subjects were excluded from qualification phase analyses due to the presence of skin extraintestinal manifestations (ie. psoriasis, erythema nodosum) as possible confounding factors. Independent age and sex-matched cohorts were used for each set of analyses. A total of 94 subjects were included in this study (Table 2).

TABLE 2
Subject Characteristics by Study.

| | | | | | Age at | | | |
|---|---|---|---|---|---|---|---|---|
| | | | | | disease | Duration | | |
| | | | | Age | onset | of disease | | ESR |
| | Cases | Male | Female | (years) | (years) | (years) | CRP (mg/L) | (mm/hr) |
| Discovery Phase | (n = 34) | (n = 17) | (n = 17) | | | | | |
| ICD | 16 | 9 | 7 | 31 ± 13 | 26 ± 21 | 6 ± 4 | 13 ± 21 | 26 ± 52.5 |
| SCD | 9 | 2 | 7 | 49 ± 23 | 32 ± 14 | 7 ± 2 | 6 ± 8 | 29 ± 17.5 |
| FCD | 9 | 6 | 3 | 27 ± 4 | 24 ± 4 | 3 ± 2 | 9 ± 5 | 28 ± 7 |
| P-value between | | | 0.133 | 0.390 | 0.203 | 0.195 | 0.698 | 0.799 |
| groups | | | | | | | | |
| Qualification | | | | | | | | |
| Phase | (n = 50) | (n = 25) | (n = 27) | | | | | |
| C | 10 | 5 | 5 | 39 ± 36 | NA | NA | 0.45 ± 0.05 | 6 ± 1 |
| UC | 10 | 5 | 5 | 31 ± 6 | NA | NA | 5.1 ± 14.3 | 21.5 ± 29.5 |
| ICD | 10 | 5 | 5 | 34 ± 14 | 31 ± 19 | 7 ± 2 | 14.6 ± 15.1 | 18 ± 14 |
| CCD | 10 | 5 | 5 | 35 ± 17 | 30 ± 9 | 11 ± 13 | 4.6 ± 4.5 | 13 ± 3 |
| RA | 10 | 3 | 7 | 50 ± 18 | NA | NA | 2.5 ± 4.3 | 13.5 ± 10.2 |
| P-value between | | | 0.863 | 0.036^{‡} | 0.531 | 0.629 | 0.165 | 0.763 |
| groups | | | | | | | | |
| Longitudinal CD | | | | | | | | |
| subanalysis | | | | | | | | |
| cohort^{€} | (n = 5) | (n = 3) | (n = 2) | | | | | |
| 1^{st} sample | 5 | 3 | 2 | 39 ± 19 | 30 ± 9 | 16 ± 13 | 4.2 ± 4.6 | 9 ± 5.2 |
| 2^{nd} sample | 5 | 3 | 2 | 39 ± 19 | 30 ± 9 | 16 ± 13 | 1.4 ± 1.1 | 10 ± 7 |

| | | | | Immuno- | | | Ileal |
|---|---|---|---|---|---|---|---|
| | CDA1/Partial | Biologies | Steroids | modulators | Mesalazines | Resection | disease |
| | MAYO/DAS28 | (N) | (N) | (N) | (N) | (N) | (N) |
| Discovery Phase | | | | | | | |
| ICD | 114.5 ± 130.7 | 5 | 8 | 8 | 14 | 7 | 5 |
| SCD | 41 ± 98.5 | 2 | 4 | 4 | 2 | 6 | 6 |
| FCD | 130 ± 41 | 6 | 3 | 6 | 5 | 4 | 8 |
| P-value between | 0.094 | 0.111 | 0.673 | 0.73 | 0.005 | 0.506 | 0.015 |
| groups | | | | | | | |
| Qualification | | | | | | | |
| Phase | | | | | | | |
| C | NA | 0 | 0 | 0 | 0 | 0 | NA |
| UC | 6.5 ± 6.3 | 0 | 7 | 7 | 6 | 0 | NA |
| ICD | 124.6 ± 147.3 | 2 | 3 | 4 | 3 | 2 | 4 |
| CCD | 159.2 ± 223 | 7 | 2 | 6 | 2 | 7 | 6 |
| RA | 2.0 ± 1.4 | 3 | 5 | 7 | 0 | 0 | NA |
| P-value between | 0.095* | 0.006^{†} | 0.110 | 0.475 | 0.060 | 0.02^{±} | 0.371 |
| groups | | | | | | | |
| Longitudinal CD | | | | | | | |
| subanalysis | | | | | | | |
| cohort^{€} | | | | | | | |
| 1^{st} sample | 142 ± 109 | 4 | 2 | 2 | 4 | 4 | 4 |
| 2^{nd} sample | 216 ± 108 | 5 | 2 | 3 | 4 | 4 | 4 |

C = Controls,
ICD = Inflammatory Crohn's,
SCD = Stricturing Crohn's,
FCD = Fistulizing Crohn's,
CCD = Complicated (SCD and FCD) Crohn's,
UC = Ulcerative colitis,
RA = Rheumatoid arthritis.
Dichotomous variables (medications, gender, resection) were compared between groups by chi-square test and continuous variables (age, age at disease onset, duration of disease,
CRP, ESR, disease activity score) by kruskal wallis.
Continuous variables are reported as median ± interquartile range.
Controls were excluded from comparisons based on disease activity, medications, and resections.
C, UC, and RA were excluded from comparisons based on age at disease onset, disease duration, and ileal disease.
^{‡}UC subjects were significantly younger than RA subjects (P < 0.01). There were no other differences in age between groups.
^{†}Frequency of biologics was significantly higher in CCD (P < 0.01).
^{±}Frequency of resections was significantly higher in CCD compared to UC and ICD (P < 0.05) (RA and C were excluded from comparison).
Only UC, ICD and CCD were compared for frequency of mesalazine therapy.
*Comparison between groups was made by categorical sorting of individual disease scores into remission, mild, moderate, and severe disease categories based on respective scoring reference ranges.
^{€}The longitudinal sub-analysis cohort consisted of CCD subjects sampled twice over 54 ± 14 days with continual active complication(s) (confirmed by radiologic/endoscopic evidence on the day of each blood draw as part of routine care).

### Inflammatory and Complicated Crohn's Disease Have Distinct Low-Mass Serum Proteomes

7,099 common ion features with MS/MS spectra were detected across all samples. A total 16,287 ms/ms spectra was available for the 7,099 features and the median within-group % coefficient of variation (% CV) for these features was 16.5%. The 7,099 features sequenced to 2,164 unique peptides (FDR=5.3%) which mapped to 348 proteins. 3960 ions (859 peptides) were significantly changing between behavioural phenotypes by MS1 ion intensity quantitation (P<0.05). After controlling for multiple testing (FDR q<0.01) and statistical power (>80% - based on sample size, signal delta (A) between groups, % CV within group), the portion of significantly changing ions was 3133 (716 peptides). This mapped to 172 identified proteins that were significantly modulated between ICD and CCD (P<0.05, q<0.01, power >80%). Protein modulation between SCD and FCD was less pronounced (N=118, P <0.05, q<0.01, power>80%).

Low-Mass Serum Proteome of Complicated Crohn's Disease Enriched with Epithelial Component Proteins

Functional annotation clustering discriminated the 172 protein dataset into 12 clusters of enrichedbiological module groups. Four groups were significantly enriched using a Benjamini-Hochberg -corrected EASE score cutoff of P<0.01. Within the 4 significant clusters the top 5 GO terms (by fold enrichment) associated with upregulated proteins in CCD were: desmosome, cornified envelope, ectoderm development, apical junction complex, and keratinocyte differentiation (BH-corrected EASE score P<0.001) (Table 3).

TABLE 3
Table 3. Enriched Gene Ontology (GO)
terms in Complicated Crohn's disease (CCD).

| | | | | Benjamini- |
|---|---|---|---|---|
| | | | | Hochberg |
| | Protein | Fold | EASE | corrected |
| GO Term | Count | Enrichment | Score | P-value |
| Desmosome | 6 | 120.0 | 7.60E−10 | 1.50E−08 |
| Cornified envelope | 4 | 75.0 | 1.70E−06 | 1.50E−04 |
| Ectoderm | 15 | 29.0 | 1.50E−17 | 7.40E−15 |
| development | | | | |
| Apical junction | 7 | 29.0 | 9.50E−08 | 1.20E−06 |
| complex | | | | |
| Keratinocyte | 5 | 29.0 | 2.10E−05 | 2.70E−03 |
| differentiation | | | | |
| Intermediate filament | 11 | 25.0 | 6.60E−12 | 3.30E−10 |
| Cell to cell junction | 8 | 17.0 | 2.20E−07 | 2.20E−06 |
| Anchoring junction | 7 | 17.0 | 2.50E−06 | 2.30E−05 |
| Epithelium | 7 | 12.0 | 1.90E−05 | 3.10E−03 |
| development | | | | |
| Cytoskeleton | 21 | 6.3 | 4.30E−13 | 4.30E−11 |

32 unique proteins of the 172 significantly changing protein dataset between Inflammatory Crohn's and CCD were annotated across the top 10 GO terms by functional cluster analysis (EASE score P < 0.05, Benjamini-Hochberg correction P < 0.01). Please note that proteins may be annotated to more than one GO term.

The 11 downregulated proteins in CCD in the 172 protein dataset were not found to have significant functional annotations. Ninety-six single peptide identified upregulated proteins in CCD that did not annotate to enriched biological module groups were discarded from further analysis and a final high-confidence dataset of 76 modulating proteins with functional annotation information was finalized.

### Desmosomal and Anti Microbial Proteins Selected for Qualification Phase Proteomics

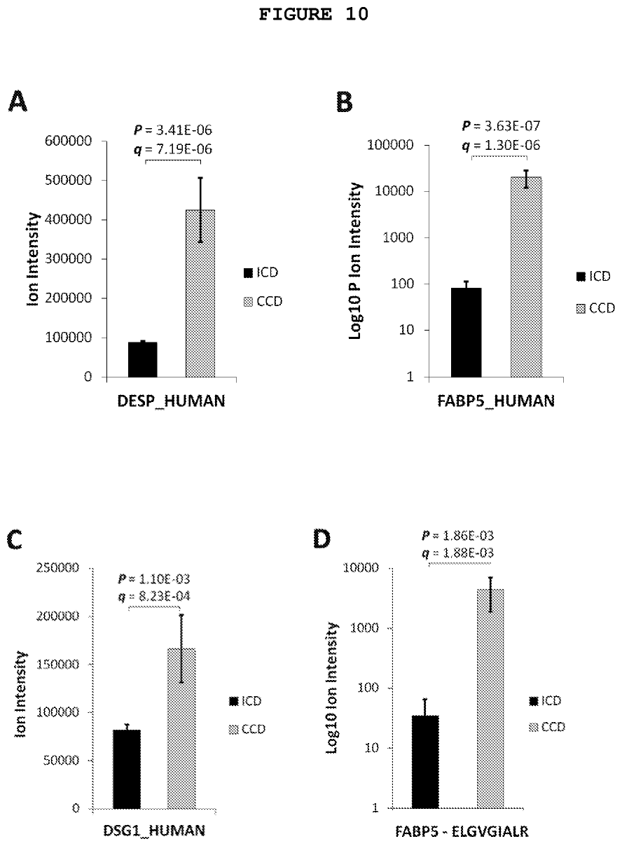

DSG1, DSK and FABP5 proteins were selected for immunoblot qualification based on significant differential regulation (P<0.01) by label-free LC-MS/MS (FIG. 10A-C) and inclusion in the significantly enriched clusters and GO terms by functional annotation analysis (BH-corrected EASE score P<0.01) (Table 3). Applying the NCI-FDA workshop statistical model, markers expressed in >80% of samples in a 16:18 case/control (ICD versus CCD) discovery phase study with a signal Δ≥5.0 standard deviations would yield >80% power in verification phase with a minimum assay N=20 (Skates et al. (2013)). The mean signal Δ for the 3 protein panel in this study was 5.1. FABP5 was further chosen for tier 2 MRM assay development based on significant differential regulation at the peptide level (P<0.01) by a high-confidence sequenced proteotypic peptide (FIG. 10D), its high discriminant function coefficient for complication (see Results—Serum Epithelial Proteins Discriminative of CCD), unique immunological and cell trauma properties (Ng EW, et al. Annals of surgery. 2013 Dec; 258(6):1111-8; Gally F, Chu H W, Bowler R P. PloS one. 2013; 8(1):e51784; Adachi et al. Histochemistry and cell biology. 2012 Sep; 138(3):397-406), and established stability in a healthy population-based cohort (Ishimura, et al. PloS one. 2013; 8(11):e81318).

### Serum Epithelial Proteins Discriminative of CCD

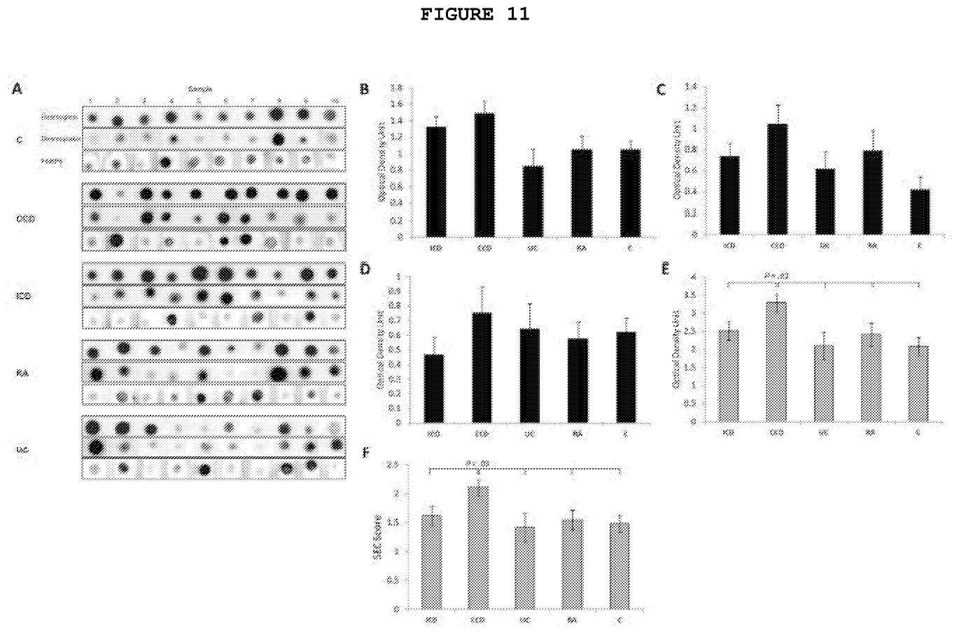

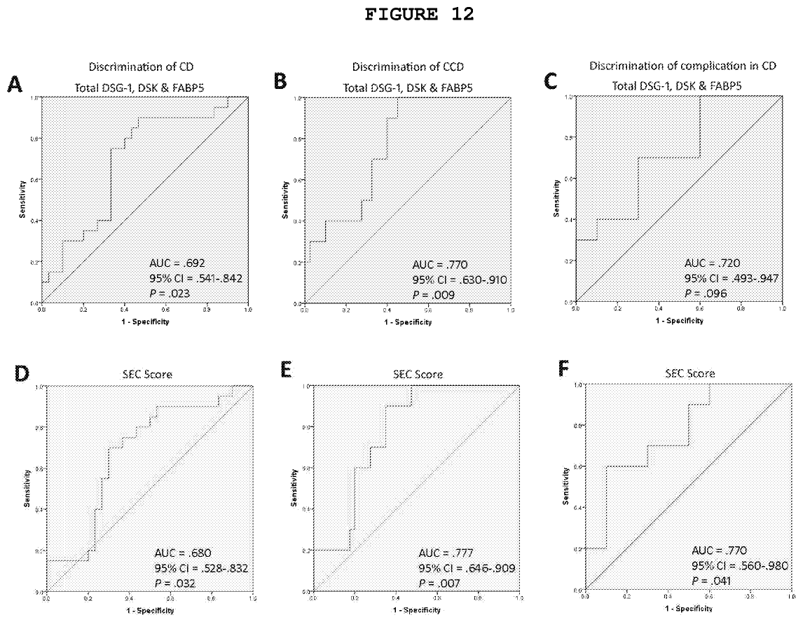

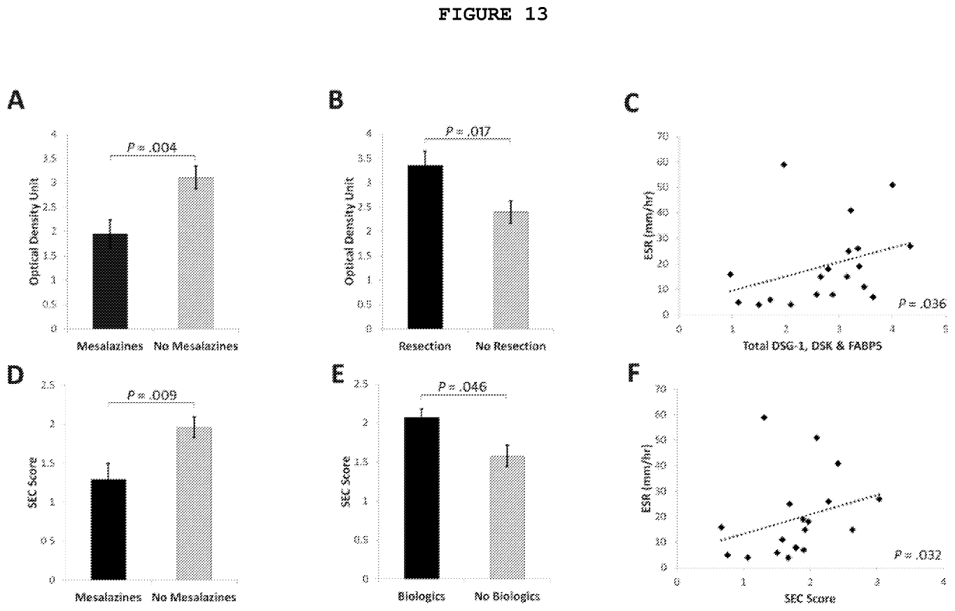

Total DSG1, DSK and FABP5 was increased in CCD (3.29±0.25 optical density unit) compared to ICD (2.52±0.26), UC (2.10±0.38), RA (2.42±0.32) and Cs (2.09±0.25) (P=0.022) (FIG. 11E). By ROC curve, total DSG1, DSK and FABP5 could significantly discriminate CD and CCD from all other conditions, and had 90% sensitivity and 60% specificity at a cutoff value of ≥2.584 for CCD discrimination (FIG. 12A-B). Classification ability of total DSG1, DSK and FABP5 for complicated phenotypes within CD did not reach statistical significance (FIG. 12C). Total DSG1, DSK and FABP5 also positively correlated with ESR (P=0.036), and was lower in those on mesalazine therapy (P=0.004) and in those without previous resection(s) (P=0.017) (FIG. 13A-C). Total DSG1, DSK and FABP5 was not associated with any other clinical factors: CRP (P=0.630), CDAI (P=0.582), biologics (P=0.120), corticosteroids (P=0.132) or immunomodulators (azathioprine/6-mercaptopurine/methotrexate) (P=0.918). Neither age (P=0.915), gender (P=0.145), age at diagnosis (P=0.667), duration of disease (P=0.931), nor ileal disease location (P=0.684) were associated with total DSG1, DSK and FABP5. ESR, mesalazine therapy, and previous resection(s) were hence entered into a multiple regression model to determine independent predictiveness for biomarker values. The model was not a significant predictor (P=0.173) of total DSG1, DSK and FABP5 values. ESR and previous resection(s) approached significance as independent predictors (P=0.069 and P=0.052, respectively), whilst mesalazines did not (P=0.938).

DSG1, DSK and FABP5 was a significant CCD classification model by discriminant function analysis (P=0.036). Standardized discriminant function coefficients of 0.726 (DSG1), 0.284 (DSK) and 0.971 (FABP5) were hence used to construct the SEC score. SEC score was significantly higher in CCD (P=0.032) (FIG. 11F) and could significantly discriminate CD (P=0.023) and CCD (P=0.007) against all other groups (FIG. 12D-E) . SEC score also improved classification of complicated phenotypes within CD (P=0.041) (FIG. 12F) with 70% sensitivity and 70% specificity at a cutoff value of 1.907. The positive and negative predictive value at this cutoff was 71.8% and 70.7%, respectively. The specificity could be improved to 90% at an SEC cutoff of 1.999, with 60% sensitivity and 86% positive and 69% negative predictive values. SEC score remained correlated with ESR (P=0.032) and lower in subjects on mesalazines (P=0.009) (FIG. 13D and F). SEC score was also significantly higher in those on biologics (P=0.046) (FIG. 13E). SEC score was not associated with any other relevant clinical factors: resection(s) (P=0.163), ileal disease (P=0.415), age (P=0.920), gender (P=0.563), steroidal therapy (P=0.340), immunomodulators (P=0.426), CRP (P=0.497), CDAI (P=0.997), age at diagnosis (P=0.373) or duration of disease (P=0.923). ESR, mesalazine therapy, and biologics was not a significant predictive model for SEC score by multiple regression (P=0.761), and neither ESR (P=0.761), mesalazine therapy (P=0.761), nor biologics (P=0.761) were independent predictors of SEC score. Individual DSG1, DSK, and FABP5 values were all increased in CCD compared to ICD, UC, RA and Cs, but the differences did not reach significance alone (P=0.051, P=0.103, P=0.708, respectively)(FIG. 11B-D).

### Serum FABP5 Levels Increased in CCD and Stable in a Longitudinal Cohort

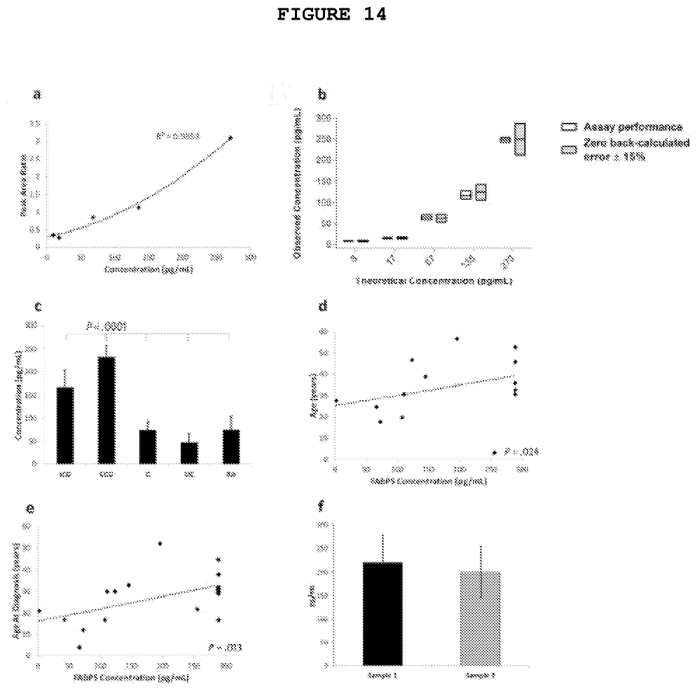

An MRM assay for FABP5 was developed that met NCI-CPTAC Tier-2 specifications(FIG. 14A-B) (Carr et al. (2014)). Increases in serum FABP5 levels in CCD identified by label-free LC-MS/MS and immunoblotting were further verified by MRM assay; with FABP5 levels higher in CCD (232.37±26.15 pg/mL) compared to ICD (167.41±36.85), UC (46.95±19.22),

RA (74.79±29.46), and Cs (74.04±18.93) (P<0.001)(FIG. 14C). FABP5 levels were moderately correlated with age (P=0.024) and age at diagnosis (P=0.013; FIG. 14D-E) but not with duration of disease (P=0.728) nor inflammation and disease activity markers: CRP (P=0.062), ESR (P=0.368) or CDAI (P=0.179). FABP5 levels alone could not discriminate CCD from ICD by ROC curve (P=0.290). There were also no differences in FABP5 based on previous resections (P=0.529), or current biologic (P=0.297), steroid (P=0.074), immunomodulator (P=0.884) or mesalazine therapy (P=0.520). FABP5 levels were additionally unrelated to gender (P=0.706) or ileal disease (P=0.647). FABP5 levels were not significantly different in five CCD subjects who gave serial blood samples (54±14 days between sampling) with continual active complication(s) (P=0.829) (FIG. 14F).

### Discussion

In this study an increase of circulating epithelial component proteins was identified in CCD using proteomics studies as part of an NCI-FDA modeled clinical biomarker pipeline. This finding supports the dysregulation of epithelial barrier function in CD. Stricturing and fistulizing intestinal complications begin with deep tissue damage following chronically sustained inflammation, thus a measure of transmural tissue integrity may be able to provide early detection of impending complication development.

Increased epithelial component proteins detected in discovery phase included major desmosomal components (DSG1, DSK, desmocollins, plakoglobin), desmosome-associated intermediate filaments (keratins, filaggrins) and immunoregulatory epithelial proteins (FABP5, Peroxiredoxin-1, Serpin B3). These proteins may be liberated in transmural intestinal injury either as a primary pathogenic mechanism or secondary consequence, and become detectable in the blood. Three epithelial components in particular, DSG1, DSK and FABP5, were confirmed to be increased in a second independent qualification cohort of CCD against ICD and UC (as intestinal inflammation controls), RA (Th1/17 systemic inflammatory control) and healthy controls. An MRM assay was then developed for FABP5 as part of the NCI-FDA biomarker pipeline that further confirmed increased levels in CCD and viability on longitudinal follow up. The panel of DSG1, DSK and FABP5, and the composite SEC score were able to discriminate CCD from ICD and all other groups. The SEC score was also independent of acute inflammation as measured by CRP, which may further indicate that it is a direct measure of barrier function. Construction of the SEC score was guided by standardized discriminant function coefficient calculations as part of a discriminant function analysis, which determines the strength and direction of each individual biomarker in contributing to the maximum discriminability of the overall model for complication in CD. This allows an objective assessment of the efficacy of each candidate to the biomarker panel, with the potential to remove candidates that negatively affect discriminability. DSG1, DSK, and FABP5 all exhibited positive coefficients for CCD discrimination (0.726, 0.284, and 0.971, respectively) and were hence included in the current working SEC score.

Increase of FABP5 in CCD was confirmed by MRM assay, and the longitudinal stability of FABP5 levels in the presence of active complications was also demonstrated. The high sensitivity and tight margin-of-error of a validated Tier 2 MRM assay allows quantification of very low concentration biomarkers and is a crucial aspect of the research assay optimization phase in the NCI-FDA biomarker pipeline. The Tier 2 assay developed in this study attained a quantification range of 1-300 pg/mL, achieving a significant practical capability that must be met in order to detect subclinical transformations that may begin with small (and previously undetectable) concentration changes. Early detection of serologically increased intestinal epithelial components may have the potential to objectively measure transmural damage that precedes clinical manifestation of complication. This may be a time-sensitive and individual-specific indicator, in contrast to clinical risk factors.

Examinations under anaesthesia and radiological/ultrasound imaging can only diagnose intestinal complications after the fact. Urine lactulose/mannitol ratio can evaluate small bowel permeability, but not the integrity of the large intestine and thus limits its sensitivity for complication. In fact, the bloodstream may be one of the only mediums capable of prognosis because of its circulation in the microvasculature, making it accessible to all layers and segments of the intestinal tract.

LC-MS/MS results were verified in a second independent cohort of sex and age-matched inflammatory and differential IBD controls using antibody based techniques. As part of qualification phase analyses, biomarker values were also evaluated against relevant clinical markers, IBD history and medication use. Total DSG1, DSK and FABP5 levels and SEC score were higher in subjects not on mezalazine therapy (FIG. 13A & D).

In conclusion, proteomic studies have identified epithelial barrier component proteins that discriminate CCD from ICD, RA, UC and controls. The biomarker panel presented here demonstrates a serological test to assess transmural disease behavior in CD. This provides an individualized indication for timely aggressive immunosuppressive therapy.

1. A method of diagnosing, assessing, or monitoring impaired gastrointestinal barrier function in a subject, comprising comparing the level of one or more markers in a tissue or body fluid of the subject relative to a reference value for the one or more markers, wherein the one or more markers are selected from the group consisting of:
i) desmoglein or a fragment thereof;
ii) desmoplakin or a fragment thereof;
iii) SPP 24 or a fragment thereof;
iv) Paraoxonase or a fragment thereof;
v) CD14 protein precursor or a fragment thereof;
vi) Nck-associated protein 1 or a fragment thereof;
vii) Claudin-1 or a fragment thereof;
viii) Claudin-3 or a fragment thereof;
ix) Fatty acid-binding protein 5 or a fragment thereof;
x) Occludin or a fragment thereof;
xi) Alpha-1-microglobulin or a fragment thereof;
xii) Guanylate cyclase activator 2a or a fragment thereof;
xiii) Serglycin or a fragment thereof;
xiv) Propanoic acid;
xv) Hydroxybutyric acid;
xvi) Citric acid;
xvii) Proline;
xviii) Valine;
xix) 4-hydroxy-benzoic acid; and
xx) Anthranilic acid.

2. The method of claim 1, wherein the one or more markers is selected from the group consisting of:
(a) desmoglein or a fragment thereof;
(b) desmoplakin or a fragment thereof;
(c) SPP 24 or a fragment thereof;
(d) CD14 protein precursor or a fragment thereof;
(e) Nck-associated protein 1 or a fragment thereof;
(f) Claudin-1 or a fragment thereof;
(g) Claudin-3 or a fragment thereof;
(h) fatty acid-binding protein or a fragment thereof;
(i) Occludin or a fragment thereof;
(j) Guanylate cyclase activator 2a or a fragment thereof;
(k) Serglycin or a fragment thereof;
(l) Propanoic acid;
(m) Hydroxybutyric acid;
(n) Citric acid;
(o) Proline;
(p) Valine;
(q) 4-hydroxy-benzoic acid; and
(r) Anthranilic acid,
wherein the reference value for the one or more markers is the level of the one or more markers in a subject not suffering from impaired gastrointestinal barrier function, and wherein impaired gastrointestinal barrier function is diagnosed when the level of the one or more markers is elevated relative to the reference value.

3. The method of claim 1, wherein the impaired gastrointestinal barrier function in a subject is leaky gut syndrome.

4. The method of claim 3, wherein the one or more markers is selected from the group consisting of:
(i) desmoglein or a fragment thereof;
(ii) SPP 24 or a fragment thereof;
(iii) desmoplakin or a fragment thereof;
(iv) Nck-associated protein 1 or a fragment thereof;
(v) CD14 protein precursor or a fragment thereof;
(vi) Fatty acid-binding protein or a fragment thereof;
(vii) Claudin-1 or a fragment thereof;
(viii) Claudin-3 or a fragment thereof;
(ix) Occludin or a fragment thereof;
(x) Guanylate cyclase activator 2a or a fragment thereof;
(xi) Serglycin or a fragment thereof;
(xii) Propanoic acid;
(xiii) Hydroxybutyric acid;
(xiv) Citric acid;
(xv) Proline;
(xvi) Valine;
(xvii) 4-hydroxy-benzoic acid; and
(xviii) Anthranilic acid,
wherein the reference value for the one or more markers is the level of the one or more markers in the tissue or body fluid of a subject not suffering from leaky gut syndrome, and wherein leaky gut syndrome is diagnosed when the level of the one or more markers is elevated relative to the reference value.

5. The method of claim 3, wherein the marker is selected from the group consisting of:
(i) desmoglein or a fragment thereof;
(ii) SPP 24 or a fragment thereof;
(iii) Nck-associated protein 1 or a fragment thereof;
(iv) Claudin-1 or a fragment thereof;
(v) Claudin-3 or a fragment thereof;
(vi) Occludin or a fragment thereof;
(vii) Guanylate cyclase activator 2a or a fragment thereof; and
(viii) Serglycin or a fragment thereof,
wherein the reference value for the one or more markers is the level of the one or more markers in the tissue or body fluid of a subject suffering from leaky gut with low leak, and leaky gut with high leak is diagnosed when the level of the one or more markers is elevated relative to the reference value.

6. The method of claim 3, wherein the marker is selected from the group consisting of:
(a) Paraoxonase or a fragment thereof; and
(b) Alpha-1-microglobulin or a fragment thereof,
wherein the reference value for the one or more markers is the level of the one or more markers in the tissue or body fluid of a subject not suffering from leaky gut syndrome, or suffering from leaky gut with low leak, and leaky gut syndrome with high leak is diagnosed when the level of the one or more markers is reduced relative to the reference value.

7. The method of claim 1, wherein the impaired gastrointestinal barrier function in a subject is complicated Crohn's disease (CCD), wherein the one or more markers are selected from the group consisting of:
(i) desmoglein or a fragment thereof;
(ii) desmoplakin or a fragment thereof;
(iii) SPP 24 or a fragment thereof;
(iv) CD14 protein precursor or a fragment thereof;
(v) Fatty acid-binding protein 5 or a fragment thereof;
(vi) Claudin-3 or a fragment thereof; and
(vii) Occludin or a fragment thereof,
wherein the reference value for the one or more markers is the level of the one or more markers in the tissue or body fluid of a subject not suffering from CCD, and wherein CCD is diagnosed when the level of the one or more markers is elevated relative to the reference value.

8. The method of claim 7, wherein the one or more markers are selected from the group consisting of:
(i) desmoglein or a fragment thereof;
(ii) desmoplakin or a fragment thereof; and
(iii) Fatty acid-binding protein 5 or a fragment thereof,
wherein the reference value for the one or more markers is the level of the one or more markers in a subject not suffering from CCD, and wherein CCD is diagnosed when the level of the one or more markers is elevated relative to the reference value.

9. The method of claim 7, wherein the reference value is the level of FABPS or a fragment thereof in a subject not suffering from CCD, and wherein CCD is diagnosed when the level of FABPS or a fragment thereof is elevated relative to the reference value.

10. The method of claim 1, wherein the tissue or body fluid is serum.

11. The method of claim 1, wherein the desmoglein or a fragment thereof comprises the amino acid sequence of SEQ ID NO: 17; and/or the desmoplakin or a fragment thereof comprises the amino acid sequence of SEQ ID NO: 18; and/or the SPP24 or a fragment thereof comprises the amino acid sequence of SEQ ID NO: 15 or 19; and/or the PON1 or a fragment thereof comprises the amino acid sequence of SEQ ID NO: 20; and/or the CD14 or a fragment thereof comprises the amino acid sequence of SEQ ID NO: 21; and/or the NCKP1 or a fragment thereof comprises the amino acid sequence of SEQ ID NO: 22; and/or the Claudin 1 or a fragment thereof comprises the amino acid sequence of SEQ ID NO: 23; and/or the Claudin 3 or a fragment thereof comprises the amino acid sequence of SEQ ID NO: 24; and/or the FABPS or a fragment thereof comprises the amino acid sequence of SEQ ID NO: 25; and/or the occludin or a fragment thereof comprises the amino acid sequence of SEQ ID NO: 26; and/or the AMBP or a fragment thereof comprises the amino acid sequence of SEQ ID NO: 27; and/or the guanylin or a fragment thereof comprises the amino acid sequence of SEQ ID NO: 16; and/or the serglycin or a fragment thereof comprises the amino acid sequence of SEQ ID NO: 14.

12.-23. (canceled)

24. A composition for diagnosing or assessing or monitoring impaired gastrointestinal barrier function in a subject, comprising:
(a) one or more markers selected from the group consisting of:
(i) desmoglein or a fragment thereof;
(ii) desmoplakin or a fragment thereof;
(iii) SPP 24 or a fragment thereof;
(iv) Paraoxonase or a fragment thereof;
(v) CD14 protein precursor or a fragment thereof;
(vi) Nck-associated protein 1 or a fragment thereof;
(vii) Claudin-1 or a fragment thereof;
(viii) Claudin-3 or a fragment thereof;
(ix) Fatty acid-binding protein or a fragment thereof;
(x) Occludin or a fragment thereof;
(xi) Alpha-1-microglobulin or a fragment thereof;
(xii) Guanylate cyclase activator 2a or a fragment thereof;
(xiii) Serglycin or a fragment thereof;
(xiv) Propanoic acid;
(xv) Hydroxybutyric acid;
(xvi) Citric acid;
(xvii) Proline;
(xviii) Valine;
(xix) 4-hydroxy-benzoic acid; and
(xx) Anthranilic acid; or
(b) one or more antibodies or antigen binding fragments thereof, wherein each antibody or antigen binding fragment thereof specifically binds a marker selected from the group consisting of:
(i) desmoglein or a fragment thereof;
(ii) desmoplakin or a fragment thereof;
(iii) SPP 24 or a fragment thereof;
(iv) Paraoxonase or a fragment thereof;
(v) CD14 protein precursor or a fragment thereof;
(vi) Nck-associated protein 1 or a fragment thereof;
(vii) Claudin-1 or a fragment thereof;
(viii) Claudin-3 or a fragment thereof;
(ix) Fatty acid-binding protein or a fragment thereof;
(x) Occludin or a fragment thereof;
(xi) Alpha-1-microglobulin or a fragment thereof;
(xii) Guanylate cyclase activator 2a or a fragment thereof; and
(xiii) Serglycin or a fragment thereof.

25. A device for diagnosing or assessing impaired gastrointestinal barrier function in a subject, comprising:
(a) one or more markers selected from the group consisting of:
(i) desmoglein or a fragment thereof;
(ii) desmoplakin or a fragment thereof;
(iii) SPP 24 or a fragment thereof;
(iv) Paraoxonase or a fragment thereof;
(v) CD14 protein precursor or a fragment thereof;
(vi) Nck-associated protein 1 or a fragment thereof;
(vii) Claudin-1 or a fragment thereof;
(viii) Claudin-3 or a fragment thereof;
(ix) Fatty acid-binding protein or a fragment thereof;
(x) Occludin or a fragment thereof;
(xi) Alpha-1-microglobulin or a fragment thereof;
(xii) Guanylate cyclase activator 2a or a fragment thereof;
(xiii) Serglycin or a fragment thereof;
(xiv) Propanoic acid;
(xv) Hydroxybutyric acid;
(xvi) Citric acid;
(xvii) Proline;
(xviii) Valine;
(xix) 4-hydroxy-benzoic acid; and
(xx) Anthranilic acid; or
(b) one or more antibodies or antigen binding fragments thereof, wherein each antibody or antigen binding fragment thereof specifically binds a marker selected from the group consisting of:
(i) desmoglein or a fragment thereof;
(ii) desmoplakin or a fragment thereof;
(iii) SPP 24 or a fragment thereof;
(iv) Paraoxonase or a fragment thereof;
(v) CD14 protein precursor or a fragment thereof;
(vi) Nck-associated protein 1 or a fragment thereof;
(vii) Claudin-1 or a fragment thereof;
(viii) Claudin-3 or a fragment thereof;
(ix) Fatty acid-binding protein or a fragment thereof;
(x) Occludin or a fragment thereof;
(xi) Alpha-1-microglobulin or a fragment thereof;
(xii) Guanylate cyclase activator 2a or a fragment thereof; and
(xiii) Serglycin or a fragment thereof.

26. The device of claim 25, wherein the one or more markers or antibodies are immobilised on a solid support.

27. The device of claim 26, wherein the solid support is selected from the group consisting of stick, plate, bead, microbead or array.

28. A kit when used for diagnosing or assessing impaired gastrointestinal barrier function in a subject, comprising:
(a) one or more markers selected from the group consisting of:
(i) desmoglein or a fragment thereof;
(ii) desmoplakin or a fragment thereof;
(iii) SPP 24 or a fragment thereof;
(iv) Paraoxonase or a fragment thereof;
(v) CD14 protein precursor or a fragment thereof;
(vi) Nck-associated protein 1 or a fragment thereof;
(vii) Claudin-1 or a fragment thereof;
(viii) Claudin-3 or a fragment thereof;
(ix) Fatty acid-binding protein or a fragment thereof;
(x) Occludin or a fragment thereof;
(xi) Alpha-1-microglobulin or a fragment thereof;
(xii) Guanylate cyclase activator 2a or a fragment thereof;
(xiii) Serglycin or a fragment thereof;
(xiv) Propanoic acid;
(xv) Hydroxybutyric acid;
(xvi) Citric acid;
(xvii) Proline;
(xviii) Valine;
(xix) 4-hydroxy-benzoic acid; and
(xx) Anthranilic acid; or
(b) one or more antibodies or antigen binding fragments thereof, wherein each antibody or antigen binding fragment thereof specifically binds a marker selected from the group consisting of:
(i) desmoglein or a fragment thereof;
(ii) desmoplakin or a fragment thereof;
(iii) SPP 24 or a fragment thereof;
(iv) Paraoxonase or a fragment thereof;
(v) CD14 protein precursor or a fragment thereof;
(vi) Nck-associated protein 1 or a fragment thereof;
(vii) Claudin-1 or a fragment thereof;
(viii) Claudin-3 or a fragment thereof;
(ix) Fatty acid-binding protein or a fragment thereof;
(x) Occludin or a fragment thereof;
(xi) Alpha-1-microglobulin or a fragment thereof;
(xii) Guanylate cyclase activator 2a or a fragment thereof; and
(xiii) Serglycin or a fragment thereof.

29. The kit of claim 28, wherein the one or more markers or antibodies is immobilised on a solid support.

30. The kit of claim 29, wherein the solid support is selected from the group consisting of stick, plate, bead, microbead or array.

31. The method of claim 1, further comprising the step of treating the subject for impaired gastrointestinal barrier function when the subject is diagnosed, assessed or monitored as suffering from impaired gastrointestinal barrier function.

32. The method of claim 31, wherein the step of treating comprises administering an effective amount of an anti-inflammatory agent, an immune modifier, or an anti-TNF agent.
